(19) United States
(12) Patent Application Publication
(10) Pub. No.: US 2008/0138319 A1
(43) Pub. Date: Jun. 12, 2008

(54) Bone-marrow derived neurogenic cells and uses thereof

(75) Inventors: Jie Deng, Durham, NC (US); Eric D. Laywell, Gainesville, FL (US); Bryon Petersen, Gainesville, FL (US); Dennis A. Steindler, Gainesville, FL (US)

Correspondence Address: EDWARDS ANGELL PALMER & DODGE LLP, P.O. BOX 55874, BOSTON, MA, 02205, US

(73) Assignee: University of Florida, Gainesville, FL (US)

(21) Appl. No.: 11/975,683

(22) Filed: Oct. 19, 2007

Related U.S. Application Data
(63) Continuation of application No. PCT/US06/14589, filed on Apr. 19, 2006.
(60) Provisional application No. 60/672,998, filed on Apr. 20, 2005.

(51) Int. Cl. A61K 48/00; C12N 5/06; C12N 5/10; A61K 35/30

(52) U.S. Cl. 424/93.2; 435/325; 424/93.7

(57) ABSTRACT

Disclosed are cellular compositions, grafts and pharmaceutical products made from bone marrow (BM) cells useful for a wide array of cell-based therapies for diseases and disorders of the nervous system. Transgenic cells in accord with the invention express therapeutic or reporter genes encoded in viral vectors. The cells are capable of extensive expansion in vitro and can generate neurons and glial cells both in culture and in vivo. Upon administration to a nervous system tissue or site in a host subject, the cells can migrate to appropriate target sites and can assimilate therein, differentiating into both neurons and astrocytes without evidence of fusion with endogenous cells of the host nervous tissue. The cells can express an array of therapeutic gene products suitable for treatment of nervous system disorders. Particularly preferred for neurodegenerative disease applications are transgenic BM-derived neurogenic cells expressing neurotrophic factors.

### CROSS-REFERENCE TO RELATED APPLICATIONS

This application is a continuation of PCT/US06/14589, filed Apr. 19, 2006, which claims the benefit of U.S. Provisional Patent Application Ser. No. 60/672,998, entitled “Bone-Marrow Derived Neurogenic Cells and Uses Thereof,” filed Apr. 20, 2005, the disclosure of which is hereby incorporated by reference in its entirety.

### STATEMENT OF U.S. GOVERNMENT INTEREST

Funding for the present invention was provided in part by the Government of the United States under Grant Nos. NS37556 and HL70143 from the National Institutes of Health. Accordingly, the Government of the United States may have certain rights in and to the invention.

### FIELD OF THE INVENTION

The invention generally relates to compositions and methods for production of cell types of the nervous tissue from stem cells. More specifically, the invention relates to the production and use of cells derived from the bone marrow of adult subjects to generate renewable sources of neural cells useful for therapies in fields of regenerative medicine, particularly for neurodegenerative disorders.

### BACKGROUND

Multipotent adult stem cells (ASC) are known to exist in a variety of adult tissues. It is believed that under appropriate conditions these cells may be able to differentiate into cell types of other lineages [1-6]. The multipotency of ASC has generated great interest in their potential therapeutic value. Unlike human embryonic stem cells (ESC), ASC do not raise ethical concerns and provide the further advantage of being relatively accessible from a patient's own body. Ongoing studies around the world are evaluating the potential of ASC to differentiate into desired cell types in a variety of adult tissues and organs [7, 8, 9-12].

One class of ASC is the so-called mesenchymal stem cell (MSC) that can be isolated from bone marrow. There is general recognition that MSC may represent a cell of choice for autologous stem cell-based replacement therapies for several reasons. In addition to their multipotency and accessibility, these cells are not expected to elicit graft-versus-host disease (GVHD) [14, 15]. For this reason, the MSC is one of the most extensively studied ASC regarding its potential to trans-differentiate into cell types of specific lineages [2, 15-18].

At present, both the distinguishing characteristics of the MSC and its qualification as a true stem cell are subject to controversy. Uncertainties arise primarily from the lack of universally defined cell surface markers to characterize the MSC as in the manner of the well-characterized hematopoietic stem cell [19-21]. Additionally, MSC isolation methodology has remained essentially unchanged for many years, and is relatively unrefined in comparison with methods in use for other cell types extensively cultivated in vitro.

The high incidence of seriously debilitating disorders of the nervous system such as Parkinson's disease, Alzheimer's disease and spinal cord injury has spurred intense interest in the ability of MSC to differentiate into cells of the neural lineage [22-24]. Several authors have described the use of chemical agents to induce a rapid and robust transformation in the appearance of MSC and other ASC to resemble neurons, whereas under normal conditions these cells exhibit a flat, fibroblast-like appearance [25-31]. There are conflicting reports, however, as to the authenticity of the so-called “neuralization” of ASC, including MSC [25, 26, 36, 40].

The capacity of the chemically treated cells to form a full range of nervous cells (both neurons and glia) is also subject to question. For example, under a protocol using the chemical agents DMSO/BHA to induce differentiation of stem cells to neural cells, expression of neurofilaments (a neuron-specific marker) is observed in MSC, suggesting that these cells are capable of differentiating into neurons. However, this apparent neurogenic capacity of MSC has been questioned in view of studies showing a similar phenomenon taking place following the same treatment of non-stem cells (i.e., epidermal fibroblasts and PC12 cells). These studies concluded that the observed neuron-like morphological and immuno-cytochemical changes of MSC were not due to bona fide neurofilament extension, but rather to cell shrinkage and retraction of the cytoskeleton in response to chemical stress, similar to changes seen in the presence of other chemicals such triton or sodium hydroxide [32, 33].

Other studies have addressed the capacity of MSC to differentiate into cells of the glial lineage. Glial fibrillary acidic protein (GFAP) is a well known marker of glial cells that has been used extensively to characterize cells of the astrocytic lineage, and to distinguish neurons from glia. The capacity of MSC to differentiate into neural and glial cells has been evaluated in a transgenic mouse line carrying a vector expressing a reporter gene under the control of a GFAP promoter [7]. From studies in vitro and in vivo, it was concluded that bone marrow-derived cells could not differentiate along the astrocytic lineage [7]. On the contrary, there have been other reports that support a conclusion of in vivo neuralization of the MSC.

Given the great potential for therapeutic use of bone marrow-derived cells in the treatment of disorders of the brain and nervous system, and the relatively early stage of scientific discovery in this important area, there is a clear need to fully elucidate the neurogenic potential of MSC. In light of disparate results from different laboratories, the need exists to fully characterize MSC using a range of cellular markers and to identify subpopulations of cells with neurogenic capacity within the bone marrow.

In order to fully realize the therapeutic potential of neurogenic bone-marrow derived cells, culture methods are needed for generating renewable sources of these cells in vitro. Further needed is clear demonstration of the capacity of neurogenic cells propagated ex vivo to home to the appropriate areas of the brain or nervous system following introduction into a host, to differentiate into neurons and glia, and to a successfully assimilate into the appropriate area of the host nervous system.

### SUMMARY OF THE INVENTION

The invention generally relates to compositions and methods for use in cell-mediated therapy including gene therapy of the nervous system using isolated bone marrow (BM) derived cells. It has been discovered that isolated BM-derived neurogenic cells (BMDNC) can be propagated in essentially limitless amounts, can differentiate into neural phenotypes in vitro (both neurons and glia) and can provide renewable sources of both neurons and glia for cellular therapy in vivo. In some instances the BMDNC are genetically modified in vitro. The invention has a wide spectrum of uses including providing unmodified or genetically modified BM cells that can be therapeutically administered to the nervous system of a patient.

More particularly, it has been discovered that under particular conditions in culture, isolated BM-derived cells express cell surface markers characteristic of mesenchymal stem cells and cells of neural lineage. We refer to these cells as “bone marrow-derived neurogenic cells” (or “BMDNC”). We show that these cells behave as stem cells, i.e., can divide symmetrically (maintaining their own self-renewal) and can generate neuron- and astrocyte-restricted progeny through asymmetric division, as further described in FIG. 4 and related text. When grafted into the lateral ventricles of the brain of a host subject, the BMDNC migrate extensively and differentiate into mature neurons and periventricular astrocytes in absence of fusion with endogenous cells of the host nervous system. The cells can be expanded extensively in vitro and continue to express appropriate cellular markers after more than 50 passages in vitro. The cells can further be transduced with viral vectors, such as a lentiviral vector, and can express a gene of interest encoded by a nucleic acid sequence contained in the vector.

Accordingly, one important aspect of the invention is an isolated neurogenic cell derived from bone marrow (BM). Under appropriate conditions in vitro, the cell can differentiate into cells that express cellular markers characteristic of neuronal or glial lineages. When administered into a recipient host subject, for example into the brain, the neurogenic cells of the invention can migrate and integrate into appropriate target regions in the brain, and can differentiate into both neurons and glia in absence of any evidence of fusion with endogenous cells of the nervous system of the subject.

Specific embodiments of the neurogenic BM-derived cells of the invention are distinguished by expression of particular combinations of cellular markers, (or in some cases absence of expression of particular markers). Neurogenic cells of the invention include but are not limited to cell types characterized by the following marker expression profile in vitro: undetectable or low levels of at least one of CD34, CD45, and CD11b; detectable levels of at least one of CD105 and CD109; undetectable or low levels of c-kit and detectable levels of sca-1; and detectable or high levels of at least one of nestin, β-III tubulin, and NFM, as determined by standard marker detection assay. Specific embodiments of the neurogenic cells of the invention are further characterized by expression of at least one marker of glial cells, such as glial fibrillary acidic protein (GFAP).

Certain embodiments of the cells are transgenic cells comprising an expression vector that includes a nucleic acid sequence encoding a therapeutic gene or a reporter gene. In a preferred embodiment, the vector is a lentiviral vector.

Also included in the invention in various embodiments are cellular compositions, grafts, and pharmaceutical products comprising at least one of the isolated neurogenic cells of the invention. One preferred embodiment is an isolated population of neurogenic cells derived from bone marrow. The cells are capable of differentiating into a neuron or a glial cell in vitro or when administered in vivo to a host subject.

The neurogenic BM-derived cells of the invention can be expanded in vitro and stored frozen until ready for use. Established cell lines of neurogenic cells made in accordance with the invention can retain their neurogenic properties after at least 50 passages in tissue culture.

The neurogenic cells of the invention can be administered to a subject in need by any suitable route using methods in accordance with the invention. In particular applications involving the central nervous system, the cells can be administered for example to the brain or spinal cord of a host subject in need of such treatment. The cells can also be administered as indicated to a component of the peripheral nervous system, such as a nerve. Upon administration to a host subject, the cells of the invention are capable of widespread distribution throughout the nervous system of the subject.

In another aspect, the invention provides a method for delivering a bone marrow-derived neurogenic cell to a neural tissue of a host subject. The method includes the steps of: (a) culturing bone marrow-derived cells under suitable conditions to obtain an isolated neurogenic cell; (b) expanding the neurogenic cell in vitro, to obtain a cell population or graft enriched in bone marrow-derived neurogenic cells; and (c) administering the cell population or graft of step (b) into the host subject. The bone marrow-derived neurogenic cell or a progeny cell thereof populates at least one neural tissue of the subject.

Another aspect of the invention is a method for expressing a therapeutic or reporter gene in a neural tissue of a subject. Practice of this method involves the use of particular embodiments of the BM-derived cells that are transgenic cells. The method includes: (a) culturing a population of BM-derived cells under suitable conditions to obtain an isolated neurogenic cell from the cell population; (b) expanding the neurogenic cell in vitro, to obtain a cell population or graft enriched in BM-derived neurogenic cells; (c) contacting the neurogenic cell of step (a) or (b) with an expression vector comprising a therapeutic or reporter gene, to obtain transgenic BM-derived neurogenic cells transduced with the vector, and (d) administering the cell population or graft of step (c) into the host subject. The neurogenic BM-derived cells are transduced with the vector in vitro, under conditions suitable for expression of the introduced therapeutic or reporter gene by the cells. If the transduction results in stable integration of the transgene into genome of the neurogenic cell, the progeny of the transduced cell will also express the transgene.

Upon administration in vivo, the transgenic neurogenic cell or a progeny cell derived from this cell can populate at least one neural tissue of the subject and express the therapeutic or reporter gene.

In some embodiments of the foregoing methods, the donor subject and the host subject are the same individual. The BM-derived cells of step (a), obtained from the host subject, are isolated, expanded and in some instances transduced with a reporter or therapeutic gene in vitro. Because the cells are from the host subject, they can be transplanted back into the host without concerns of graft rejection associated with allografts.

Another aspect of the invention is a pharmaceutical product for preventing, treating or reducing the severity of a disorder of the nervous system. The product comprises at least one of the following components: any of the above-described isolated BM-derived neurogenic cells, a graft comprising these cells, and optionally directions for preparing, maintaining and/or administering the cells or graft.

In preferred pharmaceutical products of the invention, the neurogenic BM-derived cells are transduced cells comprising a viral expression vector such as a lentiviral vector. In preferred embodiments of the foregoing isolated transgenic cells, grafts, or pharmaceutical products, the expression vector comprises a nucleic acid sequence encoding a therapeutic gene which is a neurotrophic factor. A particularly preferred neurotrophic factor for treatment of nervous system disorders is glial cell line-derived neurotrophic factor (GDNF). Other neurotrophic factors of use in certain applications include brain-derived neurotrophic factor (BDNF), neural growth factor (NGF), basic fibroblast growth factor (bFGF), and epithelial growth factor (EGF).

In yet another aspect of the invention, there is provided a method for preventing, treating or reducing the severity of a disorder of the nervous system. In one embodiment, the method includes administering to a subject in need of such treatment a therapeutically effective amount of the isolated neurogenic BM-derived cells disclosed herein.

Other aspects and advantages of the invention are discussed below.

### BRIEF DESCRIPTION OF THE DRAWINGS

FIG. 1A is six photomicrographs showing characteristics of bone marrow-derived neurogenic cells in long-term culture. The lower panel is three fluorescent images of the fields in the corresponding upper panels, showing GFP expression in BM-derived cells with fibroblast-like morphology.

FIG. 1B is nine graphs illustrating results of flow cytometric analysis of cell surface markers on neurogenic BM-derived cells after more than 50 passages in vitro, according to an embodiment of the invention.

FIG. 2A is three fluorescence micrographs showing immunolabeling of BM-derived neurogenic cells using antibodies directed against nestin, β-III tubulin, -, and neurofilament M, according to an embodiment of the invention.

FIG. 2B is a table showing quantification of expression levels of neural markers by BM-derived cells (MSC) and fibroblasts in vitro in normal culture medium (CM) and after 2-day treatment with the chemical agent dbcAMP/IBMX, according to an embodiment of the invention. The proportion (in percentages) of cells positive for each antibody is shown.

FIG. 2C is two micrographs showing that NIH3T3 fibroblast cells can acquire a neuron-like morphology after treatment with dbcAMP/IBMX for two days.

FIG. 3A is two fluorescence micrographs of cultured BM-derived neurogenic cells showing that expression of the glial marker GFAP is induced in these cells by dbcAMP/IBMX treatment, according to an embodiment of the invention.

FIG. 3B is two photomicrographs showing in situ hybridization of glial cell-specific (GFAP) transcripts in BM-derived neurogenic cells following dbcAMP/IBMX induction treatment, according to an embodiment of the invention. The inset shows immunofluorescent detection of GFAP protein in the cell indicated by the arrow.

FIG. 3C is a photograph of a Western immunoblot using GFAP monoclonal antibody to demonstrate GFAP expression in brain tissue (B, positive control) and in BM-derived neurogenic cells untreated (NT) or treated with dbcAMP/IBMX for the indicated durations in hours. Actin antibody was used as a control for protein loading.

FIG. 4A is a six fluorescence micrographs showing double immunolabeling of dbcAMP/IBMX-treated BM-derived neurogenic cells using antibodies against neuronal (βIII tubulin and NFM) and astrocytic (GFAP) markers, according to an embodiment of the invention. Cell nuclei are counterstained with DAPI.

FIG. 4B is two fluorescence micrographs (4Ba) and a schematic diagram (4Bb) illustrating that BM-derived neurogenic cells exhibit multipotency by generating progeny of different lineages through symmetric and asymmetric division. FIG. 4Ba shows representative clones immunostained with anti-neurofilament M (NFM) antibody, a neuronal marker. No NFM positive cells are observed in clones containing about 5 cells (top image, n=10) whereas the NFM marker is expressed in a small proportion of cells comprising clones of more than 10 cells (n=13). An enlarged view of a NFM-expressing cell is shown in the inset. The diagram in FIG. 4Bb illustrates a model of the division (symmetric and asymmetric) and marker expression characteristics of BM-derived neurogenic cells.

FIG. 5A is five fluorescence micrographs showing that BM-derived neurogenic cells can differentiate into neurons and astrocytes upon transplantation into the lateral ventricle of the brain, according to an embodiment of the invention. Transplanted DiI-labeled BM-derived neurogenic cells exhibit morphological characteristics of typical of astrocytes in the sub-ventricular zone (SVZ; a), and typical granule interneurons in the granule cell layer of the olfactory bulb (OLB, b). Images on the right show higher magnification detail of the framed areas on the left. c: Two DiI labeled cells are also positive for Y-chromosome painting, indicating their donor cell origin.

FIG. 5B is a series of double immunfluorescence micrographs showing immunophenotypic profiles of BM-derived neurogenic cells transplanted into the brain, according to an embodiment of the invention. BM-derived neurogenic cells are seen to express neuron-specific markers β-III tubulin and PSA-NCAM, and astrocyte-specific marker GFAP. Insets in each picture show individual fluorescence channels.

FIGS. 6A and 6B are a series of fluorescence photographs taken by confocal scanning microscopy demonstrating immunolabeling of transplanted BM-derived neurogenic cells with antibodies to neuron-specific proteins. DiI-labeled BM-derived cells are immunolabeled with PSA-NCAM (6A) and β-III tubulin (6B). The images on the left are merged confocal images in the ganglion cell layer, GCL (6A) and rostral migratory stream, RMS (6B) of the olfactory bulb. The images on the right show separate fluorescence channels of the cells indicated in the merged view, demonstrating that immunofluorescence (green and red) is co-localized to the same plane.

FIGS. 7A and 7B are a series of confocal scanning microscopic photographs used to exclude the possibility of cell fusion between donor and host cells in recipients of BM-derived neurogenic cells.

FIGS. 8A and 8B are two fluorescence micrographs showing expression of a gene of interest (fluorescent signal) in BM-derived neurogenic cells transduced with a lentiviral expression vector, according to an embodiment of the invention.

### DETAILED DESCRIPTION OF THE INVENTION

Bone marrow (BM) has long been recognized as a rich source of many types of stem/progenitor cells. Those that give rise to blood cells (hematopoietic stem cells, HSC) have been most extensively characterized. Under appropriate conditions certain hematopoietic stem/progenitor cells divide and differentiate along recognized pathways to form blood cells, such as those of the erythroid, myeloid, and lymphoid lineages.

As discussed, there has been increasing appreciation that BM from adult subjects is not restricted to production of new blood cells, but is a source of multipotent stem/progenitor cells with potential to give rise to cells that differentiate into many other lineages. The invention relates to the isolation, ex vivo production and expansion of neurogenic cells derived from bone marrow (BM), and their use in cellular therapy for diseases and conditions of the nervous system. As further described below, some embodiments of the BM-derived neurogenic cells (“BMDNC”) are transgenic cells. The transgenes expressed by the BMDNC can include a wide array of therapeutic or reporter genes. Accordingly the BMDNC cells of the invention have a wide spectrum of important uses. The transgenic BMDNC can be used for a variety of therapeutic purposes, including but not limited to use in the prevention, treatment or alleviation of symptoms associated disorders and injuries of the nervous system, spinal cord, and components of the peripheral nervous system.

### Bone Marrow-Derived Neurogenic Cells (BMDNC)

The bone marrow-derived neurogenic cells of the invention include isolated cells derived from bone marrow (BM). As used herein, a “cell derived from bone marrow” (whether or not transgenic) is meant to refer to any cell type that is either: 1) directly obtained from the BM of an animal, or 2) the product of a cell that is directly obtained from the BM. Examples of the former types of cells, in particular various characterized stem/progenitor cells in the BM, are further described infra. A “cell derived from bone marrow” can also refer to a cell of the second type described above, for example a progeny cell that arose by division and/or differentiation of a cell type of the BM, including those that arise, for example, in a part of the body remote from the BM upon introduction into a host tissue, such as in the nervous system. As discussed, well known examples of such progeny of BM cells include: red blood cells (the differentiated product of BM stem/progenitor cells of the erythroid lineage); various nucleated blood cells (granulocytes) including polymorphonuclear leukocytes, eosinophils, basophils, macrophages and monocytes (differentiated cells produced by BM cells of the myeloid lineage) and lymphocytes (differentiated cells of the BM lymphocytic lineage). Other cells “derived from bone marrow” include any of the progeny cells that arise by division and/or differentiation of a “multipotent stem/progenitor cell” of the BM, including but not limited to BM-derived cells that differentiate into cells of the neuronal and glial lineages.

A “transgenic cell derived from bone marrow,” as used herein, refers to either: 1) a cell isolated from BM and transduced with an expression vector according to the invention, or 2) a transgenic cell that is the product (progeny) of such a cell.

“Isolated,” as it is used herein, refers to BM cells and BM-derived cells in vitro, or in the form of a pharmaceutical product, that have been separated from BM and other substituents that naturally accompany it. Preferably, the BM or BM-derived cells of the invention are at least 80% or 90% to 95% pure (w/w). BM-derived neurogenic cells having at least 98 to 99% homogeneity (w/w) are most preferred for many pharmaceutical, clinical and research applications. Once substantially purified or isolated, the BMDNC would be substantially free of unwanted marrow contaminants. Once purified partially or to substantial purity, the BMDNC are suited for therapeutic or other uses such as those provided herein. Purity can be determined by a variety of standard techniques such as cell culture, microscopic and centrifugation techniques (e.g., Ficoll gradient) and cell sorting methods such as fluorescence activated cell sorting (FACS), for detection and/or selection of cells bearing particular markers of cell lineage.

Whole BM aspirates may be used as starting material for BM-derived cultures of the invention. The art is well advanced in the areas of isolation of BM from bones of host subjects, tissue culture methods for propagation of BM cells, and of subpopulations of BM cells enriched in particular lineages, providing many options for selecting a starting population of BM cells to be selected for neurogenic potential in accordance with the invention. Depending on the purpose, in many applications it may be preferable to use an enriched population of BM cells as the starting material for generation of BMDNC cultures.

BM cells can be isolated for example by flushing the cells from long bones such as the femur or tibia. Mononuclear cells in the BM can be isolated by gradient centrifugation and cultured, for example as described in [34-37] and Examples infra.

As discussed, in some applications it is desirable to select and enrich a particular cell type of the BM before expansion in vitro. One useful method for obtaining a selected population of isolated BM cells involves clonal expansion, which can include at least one of and preferably all of the following process steps:
a) collecting BM cells from a mammal which cells have a size of less than about 50 microns, preferably less than about 20 microns,
b) culturing (expanding) the collected cells in medium under conditions that select for adherent cells,
c) selecting the adherent cells and expanding those cells in medium to semi-confluency,
d) serially diluting the cultured cells into chambers with conditioned medium, the dilution being sufficient to produce a density of less than about 1 cell per chamber to make clonal isolates of the expanded cells; and
e) culturing (expanding) each of the clonal isolates and selecting chambers having expanded cells to make the population of isolated BM cells.

Depending upon the type of cell desired for transduction, suitable protocols and culture conditions can be used to culture and expand populations comprising a single desired cell type.

Certain neurogenic cells of the invention are isolated stem or progenitor cells of the BM. In general, the term “stem cell” refers to an unspecialized human or animal cell that has the capacity to produce mature specialized cell types of the body and at the same time replicate (renew) itself by symmetric division. Stem cells derived from embryos (“embryonic stem cells,” “ESC”) are obtained from a blastocyst, a very early embryo that contains about 200 to 250 cells and is shaped like a hollow sphere. The embryonic stem cells in the blastocyst are the cells that ultimately would develop into a person or animal if the blastocyst were to proceed to develop into an animal. The similar “embryonic germ line cells” can be obtained from a fetus that is 5 to 9 weeks old and are derived from tissue that would have developed into the ovaries or testes.

By contrast, “adult stem cells,” “ASC” are present in several tissues of adult individuals, and can be obtained from sources such as the umbilical cord and placenta after birth, or from the peripheral blood, bone marrow, skin, and other tissues of adults. “Progenitor cells” are stem cells that are committed to differentiate along a more restricted lineage, for example to form various cell types of the nervous tissue, such as neuronal cell types (neurons, interneurons, ganglion cells, etc.) and glial cell types of nervous tissue (including astrocytes, oligodendrocytes and microglia).

The term “neurogenic,” as used herein, is meant to refer to a cell having the capacity or propensity to differentiate into one or more cell types of the nervous system or a nervous tissue, including both neuronal cell types and glial cell types.

The invention takes advantage of the rich population of naturally occurring stem or progenitor cells of the BM, which include but are not limited to the following recognized cell types: mesenchymal stem cells (MSC); human marrow stromal cells [36]; and multipotent adult progenitor cells (MAPC) as described by Jiang et al. [37]. As discussed in Examples below, it is believed that neurogenic stem and progenitor cells of the BM and in BM cultures may be heterogeneous in nature, and are not yet fully characterized with respect to their ability to form particular cell types of the neural lineage.

A particularly preferred isolated BM-derived neurogenic cell according to the invention is a BMDNC having the following marker expression profile in vitro:

undetectable or low levels of at least one of CD34, CD45, and CD11b;

detectable levels of at least one of CD105 and CD109;

undetectable or low levels of c-kit and detectable levels of sca-1; and

detectable or high levels of at least one of the neuronal markers nestin, β-III tubulin, and NFM, as determined by standard marker detection assay.

As indicated, these BMDNC express markers characteristic of MSC of BM (low CD34, C45, and CD11b, and detectable CD105 and CD109), as well as markers of the neuronal lineage (e.g., nestin, β-III tubulin, and NFM).

By the phrase “standard cell marker detection assay” is meant a conventional immunological or molecular assay formatted to detect and optionally quantitate one or more of the foregoing cell markers. Examples of conventional molecular assays for cell marker detection include well known techniques such as polymerase chain reaction (PCR) (quantitative, real-time, etc.), Northern blotting, in situ hybridization and similar techniques. See also Sambrook et al. in Molecular Cloning: A Laboratory Manual (2d ed. 1989); and Ausubel et al., Current Protocols in Molecular Biology, John Wiley & Sons, New York, 1989, for general disclosure relating to recognized immunological and molecular assays that can be used to detect cell markers.

Examples of conventional immunological assays to detect expression of protein markers include Western blotting, ELISA, RIA and fluorescence activated cell sorting (FACS). FACS is an automated or semi-automated method that is preferred for detection of markers, particularly on the cell surface, in applications calling for larger amounts of neurogenic cells.

Preferred antibodies for use in immunological assays are provided in Examples below. See generally, Harlow and Lane in Antibodies: A Laboratory Manual, CSH Publications, N.Y. (1988), for disclosure relating to these and other suitable assays.

The BM-derived neurogenic cells of the invention can be propagated in vitro using standard culture conditions. As defined herein, “standard culture conditions” refer to culture conditions suitable for the maintenance and propagation of BM cells and BMDNC without components added to stimulate these cells to differentiate along a particular lineage, for example the neural lineage. Standard culture conditions for cultivating BM cells, including methods for generating clonal cultures have been developed [34-37]. Preferred methods for isolating and culturing the specific embodiments of the BMDNC of the invention are described in detail in Example 1, infra.

Another particularly preferred BMDNC in accord with the invention further expresses detectable or high levels of at least one glial cell marker in addition to the above-described markers expressed by BMDNC cultivated under standard culture conditions.

The latter embodiments can be obtained by subjecting a BMDNC cultivated under standard conditions to a protocol that increases intracellular cAMP levels. To achieve this phenotype, certain chemical agents known to increase intracellular cytoplasmic ATP levels and to stimulate the appearance of a neuronal phenotype in cultured cells can be used. Although as shown herein, these agents have no effect on the expression of neuronal markers in BM-derived cells, they are effective in specifically upregulating the expression of glia-specific markers such as GFAP. (See Examples 4 and 5, infra). Populations of BMDNC enriched in cells of the astrocytic lineage can be made by supplementing the standard culture conditions with a chemical agent that increases ATP concentration in the cells. Such chemical agents include but are not limited to dimethylsulfoxide (DMSO), butylated hydroxyanisole (BHA), butylated hydroxytoluene (BHT), dibutyrl cyclic AMP (dbcAMP), isobutylmethylazanthine (IBMX) and combinations of these agents. A particularly preferred supplement to the standard culture medium for induction of glial-specific markers in BMDNC is a combination of dbcAMP and IBMX (dbcAMP/IBMX).

Embodiments of the BMDNC that express glial markers in accordance with the invention may be particularly useful in the treatment of diseases and conditions involving defects in glial cells. The bone marrow-derived glia themselves (i.e., culture-derived cells without genetic engineering) can provide neurotrophic, neuroprotective, and/or neuro-supportive functions that benefit host neurons and glia following grafting. The cells themselves can replace glia lost to injury or disease. Alternatively or in addition, unmodified cells according to the invention, through release of their own glial-secreted factors (some of which are natural glial growth factors), may be protective to at-risk populations of CNS neurons and glia. Thus the bone marrow-derived glia may be naturally trophic and tropic.

These and other beneficial properties of the cells render them particularly useful for the treatment of neurodegenerative diseases such as multiple sclerosis, infectious diseases including AIDS, and other diseases that result in the loss of astrocytes and other types of CNS cells including but not limited to Parkinson's disease, Alzheimer's disease, Lewy body disease, amyotrophic lateral sclerosis, multiple system atrophy, progressive supranuclear palsy, corticobasal degeneration, spinal cord injury, stroke, and paralysis.

Transgenic Bone Marrow-Derived Neurogenic Cells (BMDNC)

Some embodiments of the BM-derived neurogenic cells are transgenic cells transduced with an expression vector that comprises a therapeutic or reporter gene. Preferably the cells are transduced with a viral vector. In general, the BMDNC of a donor subject are genetically modified by contacting a population of isolated BMDNC derived from the donor with an expression vector.

As used herein, the term “transgene” refers to a heterologous gene, or recombinant construct of multiple genes (“gene cassette”) in a vector. A “transgenic cell” is a cell into which a vector comprising a transgene has been introduced. The terms “transduced,” “transduction,” and the related terms “transformed,” “transformation,” “gene transfer” and the like as used herein refer to process of being made transgenic, or the state of being transgenic. In some contexts, the terms can be used synonymously. For example a “transgenic cell” can also be referred to as a “transduced cell.” A transduced cell can also refer to a cell infected with a viral vector such as a lentiviral vector. A “stably transduced” or “stably transformed” cell refers to a cell in which the transgene is stably integrated into the genome of the cell and is accordingly passed on to daughter cells by division.

By the term “vector” is meant a recombinant plasmid or viral construct used as a vehicle to introduce one or more transgenes into a cell. Preferred vectors for in vivo use in subjects are viral vectors, and as discussed, particularly preferred viral vectors are lentiviral vectors. As used herein, “vector” is a term referring to a sequence of genetic material into which a nucleotide sequence (or “transgene,” typically a fragment of a DNA encoding a full length or partial polypeptide of interest) has been inserted, and which can be used to introduce exogenous genetic material into a cell or into the genome of an organism. An “expression vector” is vector used to introduce a DNA or RNA sequence into a cell, causing the product of the DNA or RNA (typically a protein or polypeptide) to be produced by the cell.

Typically a mammalian expression vector utilizes a promoter operably linked to the transgene to express the corresponding mRNA that can be translated to the corresponding protein or polypeptide in the cell. As used herein, a “promoter” refers to a DNA sequence to which RNA polymerase binds to initiate transcription of messenger RNA, and to which other regulatory elements bind to facilitate, regulate, enhance or suppress transcription. A promoter that is “operably linked” to a DNA sequence encoding a gene or a fragment thereof in a vector causes the DNA sequence to be expressed or produced when the vector is introduced into a cell or is provided with suitable substrates and conditions in vitro. A promoter in accord with the invention can be a “ubiquitous” promoter active in essentially all cells of a host organism (such as a human), for example, a CMV, beta-actin or optomegalovirus promoters, or it may be a promoter whose expression is more or less specific to the target cell or tissue.

An example of a useful promoter which could be used to express a gene of interest in accordance with the invention is a cytomegalovirus (CMV) immediate early promoter (CMV IE) (Xu et al., Gene 272: 149-156, 2001). These promoters confer high levels of expression in most animal tissues, and are generally not dependent on the particular encoded proteins to be expressed. Examples of other such promoters of use in the invention include Rous sarcoma virus promoter, adenovirus major late promoter (MLP), Herpes Simplex Virus promoter, HIV long terminal repeat (LTR) promoter, beta actin promoter (Genbank # K00790), or murine metallothionein promoter (Stratagene San Diego Calif.). Examples of tissue- or cell-specific promoters useful for expression of transgenes in neural tissues include the glial fibrillary acidic protein (GFAP) promotor [53], and the Tau promoter [54].

As discussed, transfection refers to a process of delivering heterologous DNA such as a viral vector encoding a transgene of interest, or plasmid DNA to a cell by physical or chemical methods. The DNA is transferred into the cell by any suitable means such as electroporation, calcium phosphate precipitation, or other methods well known in the art. Use of the term “transduction” encompasses both introducing the gene or gene cassette into a cell for purposes of tracking (as with a reporter gene), or for delivering a therapeutic gene or correcting a gene defect in a cell. Transduction in the context of producing viral vectors for gene therapy (for example lentiviral vectors) in a cell can also mean introduction of a gene or gene cassette into a producer cell to enable the cell to produce the lentiviral vector.

As discussed above, typical transgenes comprise a heterologous gene sequence, or a recombinant construct of multiple genes (“gene cassette”) in a vector. The viral vectors of the invention can be produced in vitro by introducing gene constructs into cells known as producer cells. The term “producer cell” refers one of many known cell lines useful for production of viral vectors into which heterologous genes are typically introduced by viral infection or transfection with plasmid DNA. As used herein, the term “infection” refers to delivery of heterologous DNA into a cell by a virus. Infection of a producer cell with two (or more) viruses at different times is referred to as “co-infection.”

A particularly preferred vector for the purpose is a lentiviral vector. A preferred vector uses the human elongation factor (hEF) promoter to drive enhanced GFP expression. Producer cell lines such as 293T cells can be co-transfected with the helper and transducing plasmids. In addition, HEK293 cells can be transfected by Superfect (Qiagen) following the manufacture's protocols for harvesting in large quantity. Transfection protocols as described can generate vector titers in the range of 10⁷ to 10⁸/ml in 293T cells.

### Methods of Use of BM-Derived Neurogenic Cells

In one aspect, the invention provides methods for delivering a BM-derived neurogenic cell to a neural tissue of a host subject. The methods involve the use of BM-derived neurogenic cells from a donor subject, prepared as described above. In the method, BM cells are obtained from a donor subject, neurogenic BM-derived cells are isolated therefrom and propagated in vitro and the BMDNC are subsequently administered to a recipient (host) subject, for example by transplantation of a cell population or a graft of cells.

A “donor” is defined as the source of the BM cells or BMDNC whereas a “recipient” or “host” is the subject that receives the cells or graft. Immunological relationship between the donor and recipient can be allogenic, autologous, or xenogeneic as needed. In preferred invention embodiments, the donor and recipient will be genetically identical and usually will be the same individual (syngeneic). In this instance, the graft will be syngeneic with respect to the donor and recipient. In the case of syngeneic transplantation, the BM cells are manipulated ex vivo (typically including in vitro expansion of the cell numbers before and/or after gene transfer) and then re-introduced into the donor subject.

The term “graft,” as used herein, includes (or in some embodiments consists of) the isolated BMDNC described herein. A “graft” can also refer to a cell or tissue preparation that includes BMDNC and optionally other cell types from a mammal that promote a favorable neurogenic outcome.

By “graft” is also meant BMDNC of the invention which have been administered to a recipient and become part of one or more tissues or structures of the nervous system of that recipient. In some cases the word “engraftment” will be used to denote intended assimilation (incorporation) of the BMDNC into a targeted neural tissue. Preferred engraftment involves assimilation of the cells into target neural tissue sites in the brain, spinal cord or peripheral nerve. Particularly preferred engraftment involves assimilation of the cells into these sites without fusion of the donor cells with neural or other endogenous cells of the host tissue.

A graft of the invention may also take the form of a tissue culture preparation in which the BMDNC of the invention have been combined with other cells and/or factors to promote differentiation and/or cell replication that produces an intended graft. If desired, the preparation can be combined with synthetic or semi-synthetic fibers to give structure to the graft. Fibers such as Dacron, Teflon or Gore-Tex are preferred for certain applications.

A particular example of a graft of the invention is a preparation of transgenic BMDNC that have been prepared from BM of a donor and genetically modified to prevent, treat or reduce the severity of a disorder of the nervous system. The graft preparation can include a pharmaceutically acceptable carrier such as saline and optionally factors intended to assist an intended engraftment result.

The BMDNC of the invention can be administered to the nervous system of a host subject by any medically acceptable means. In preferred embodiments, the cells are administered to the brain, spinal cord or a component of the peripheral nervous system of a subject.

Another aspect of the invention involving transgenic BMDNC is a method for expressing a therapeutic or reporter gene in a neural tissue. The method includes the following general steps:

(a) culturing a population of bone marrow-derived cells under conditions to obtain an isolated neurogenic cell from the cell population;

(b) expanding said neurogenic cell in vitro, to obtain a cell population or graft enriched in bone marrow-derived neurogenic cells;

(c) contacting the neurogenic cell of step (a) or (b) with an expression vector comprising a therapeutic or reporter gene, to obtain transgenic bone marrow-derived neurogenic cells transduced with said vector; and

(d) administering the transduced cell population or graft of step (c) into a neural tissue of the host subject, wherein a transgenic neurogenic cell or a progeny cell derived therefrom populates at least one neural tissue of the subject and expresses the therapeutic or reporter gene. Steps (a)-(d) of the method are generally carried out as described above.

As mentioned, it is an object of the present invention to provide transduced BMDNC cells useful to prevent, treat or reduce the severity of diseases or disorders affecting the nervous system. The transgenic cells of the invention can be used as “cellular gene therapy vectors” that when introduced into the nervous system of a host can track to an appropriate site and locally deliver a desired therapeutic gene product. The BMDNC of the invention, when introduced into a host animal, can be followed and distinguished from native cells of the host by virtue of labeling with a tracer such as DiI prior to transplantation, or in transgenic embodiments by detecting expression of a reporter protein such as green fluorescent protein.

As mentioned, it is an object of the present invention to provide transduced BMDNC that express a therapeutic gene to prevent, treat or reduce the severity of a nervous system disorder. Therapeutic genes of interest for use in the nervous system that can be included in expression vectors carried by the BMDNC of the invention are myriad, and can include but are not limited to neurotrophic factors such as glial-derived neurotrophic factor (GDNF), BDNF, NGF, bFGF and EGF, which have been shown to be important in protecting neurons from death in various neurodegenerative diseases including Parkinson's Disease and Alzheimer's Disease. A particularly preferred neurotrophic factor expressed by cells in accordance with the invention for treatment of Parkinson's disease is GDNF. GDNF has been shown to have a protective effect on dopaminergic neurons following direct gene delivery to the nigrostriatal region of the brain [55].

Typical numbers of BMDNC (either non-transgenic or transgenic) to use for engraftment into a neural tissue site in a host subject will depend upon recognized parameters including the particular nervous system disease or disorder to be treated. However, for most applications between from about 10³ to about 10⁷ BMDNC will suffice, and typically about 10⁵ of such cells. Cells may be administered by any acceptable route including suspending the cells in saline and administering same with a needle, stent, catheter or like device. The foregoing administration protocols will be generally suitable for most therapeutic methods disclosed herein.

### EXAMPLES

The invention is further illustrated by reference to the following non-limiting examples.

### Example 1

### Materials and Methods

1. BMDNC Culture. C57/B6 and C57/B6GFP adult mice (8 weeks) were used to establish BMDNC cultures, utilizing the physical property of plastic adherence [34, 35]. Briefly, mice were given a lethal dose of Phenobarbital, and the tibias and femurs were removed. A 22-gauge needle filled with Dulbecco's Modified Eagle's Medium (DMEM) was used to flush out whole bone marrow. The recovered cells were then mechanically dissociated, filtered through a 70 μm mesh, and plated in 35 mm tissue culture dishes containing DMEM supplemented with 20% fetal bovine serum (FBS), 0.5% gentamicin, and 1000 units/mL of Leukemia Inhibitory Factor (LIF), as per Jiang, et al. [37]. After 24 hrs, the non-adherent cells were removed, and the culture medium was completely replaced. At confluency, BMDNC were passaged (1:3 dilution) twice a week with fresh medium.

In order to generate clonal cultures, we grew single BMDNC in conditioned medium collected from confluent BMDNC cultures. Conditioned medium was centrifuged at 2,600 g for 10 mins, and filtered through a 0.22 μm mesh to eliminate cellular components. We created a dilution series with BMDNC to reach a cell density of one to two cells per 5 μL, and plated 5 μL of the cell suspension in each well of a 48-well plate. Immediately after plating, we examined each well with phase microscopy, and excluded those wells containing more than one cell. We then added 100 μL of mixed medium (50% conditioned medium +50% fresh medium). In order to ensure single-cell clonality, we again examined each well after an additional 24 hours, and discarded those containing more than one cell. Clonal BMDNC cultures were maintained in the mixed medium until confluent, at which point the cells were maintained in fresh, non-conditioned medium.

2. FACS Analysis of BMDNC. Immunofluorescence with a variety of antibodies against surface antigens was used to characterize BMDNC: directly-conjugated anti-Sca1, anti-CD34, anti-CD45, and directly-conjugated anti-mouse IgG₂ₐ (PharMingen; 1:500), used as control. In addition, the following unconjugated antibodies were used: anti-c-Kit, anti-CD9, anti-CD31, anti-CD105 (PharMingen; 1:500), and anti-CD11b (Serotec; 1:300). Primary antibodies were applied for 30 mins at room temperature (RT), followed by washing and application of fluorescent-conjugated secondary antibodies for an additional 30 mins. for the unconjugated antibodies. Cells were then centrifuged at 200 g, and washed twice in PBS to eliminate unbound antibodies. Approximately 10⁶ cells/mL cell suspension was run through a flow cytometer (CELLQuest, Becton Dickinson FACScan).

3. Immunolabeling and Cell Quantification. Immunolabeling was performed on BMDNC's plated on glass coverslips. Cells were fixed in ETOH:acetic acid (95:5) for 15 mins., washed with PBS containing 0.1% Triton-X 100 (PBST), and blocked for 30 mins. in PBST supplemented with 10% FBS. Cells were then incubated with primary antibodies overnight at 4° C., washed, and incubated in secondary antibodies for 1 hr. at RT. Cell quantification was performed under a fluorescence microscope (Olympus BX51). The ratios of positive cells were obtained by averaging three different experiments for both control and treatment groups. In each experiment, five randomly chosen views were counted and averaged.

Free-floating, 40 μm brain sections were immunolabeled with the following antibodies, as previously described [27]: nestin (Developmental Studies Hybridoma Bank, University of Iowa; 1:50), glial fibrillary acidic protein (GFAP; from Immunon; 5 drop/0.5 mL), neurofilament medium subunit (NFM; EnCor Biotech. Inc.; 1:500), βIII-tubulin (Promega; 1:1500), S100β and MAP2ab (Sigma; 1:500), Polysialylated-NCAM (PSA-NCAM; Chemicon; 1:100). Confocal laser scanning microscopic analysis of immunolabeling was done on the University of Florida Cancer Center's Leica TCS SP2 confocal laser imaging system (Leica Microsystems, Wetzlar, Germany). Every fifth sagittal section through the forebrain was selected for quantification of β-III tubulin positive cells in the hemisphere receiving cell engraftment. Total numbers of cells for the hemisphere was then obtained by multiplying by 5.

4. Neural Induction by Elevating Cytoplasmic cAMP. The protocol used for neural induction by elevating intracellular cAMP was modified from Deng, et al. (2001) [36]. In addition to primary induction medium (0.5 mM isobutylmethylxanthine (IBMX)/1 mM dibutyryl cyclic AMP (dbcAMP) (Sigma) in DMEM/F12) used for the first 24 hrs of treatment, a cocktail of growth factors (10 ng/mL of Brain-Derived Neurotrophic Factor (BDNF; Pepro Tech.), Nerve Growth Factor (NGF; Invitrogen), Epidermal Growth Factor (EGF; Pepro Tech) and basic Fibroblast Growth Factor (bFGF; Pepro Tech), and N2 Supplements (Gibco) was added to the primary induction medium for treatments longer than 24 hrs.

5. In situ Hybridization for GFAP mRNA. To generate GFAP riboprobes, we used RT-PCR to amplify a 401 bp DNA fragment of the GFAP gene (gi: 26080421) from mouse brain tissue with a pair of primers designed using the Primer 3 program (Whitehead Institute and Howard Hughes Medical Institute):

| | | |
|---|---|---|
| forward: | 5′-GCCACCAGTAACATGCAAGA-3′; | (SEQ ID NO:1) |
| reverse: | 5′-ATGGTGATGCGGTTTTCTTC-3′. | (SEQ ID NO:2) |

The PCR product was then cloned into the PCR4 TOPO vector (Invitrogen). After linearization, plasmids extracted from clones of both directions were used as templates to synthesize digoxigenin (DIG)-labeled GFAP sense and antisense probes using T7 RNA polymerase. In situ hybridization followed the protocol of Braissant and Wahli (1998) [38] with small modifications. The probe concentration was 400 ng/mL and the hybridization temperature was set at 45° C.

6. Western Blotting. For Western blotting, approximately 20 μg of protein from cell lysates was electrophoretically separated by 8% SDS-PAGE. After transfer to a nitrocellulose membrane, we applied anti-GFAP (Immunon; 1:30) antibody, and a chemiluminescence method for detection (ECL, Amersham). We then incubated the membrane in striping solution at 56° C. for 30 mins, and incubated it again using anti-actin (Abcam; 1:2,000) antibody.

7. Transplantation of BMDNC into Neonatal Mouse Brain. BMDNC were trypsinized and labeled with the fluorescent carbocyanine dye, DiI (Molecular Probes), as previously reported [39]. Briefly, cells were centrifuged for 5 mins at 1000 rpm, and resuspended in fresh medium. DiI was dissolved in absolute ethanol (2.5 mg/mL), and added to the cell suspension at a final concentration of 40 μg/mL. The cells were incubated in the DiI-containing medium for 30 mins at 37° C. before being washed three times in PBS.

DiI-labeled BMDNC were transplanted into the lateral ventricle of postnatal day 1-4 wild-type C57BL6 mice according to a protocol adapted from Laywell et al [39]. Under hypothermia anesthesia, approximately 1×10⁵ BMDNC's in 1 μL of PBS were injected into the left lateral ventricle. After 10 days survival, mice were euthanized with an overdose of Avertin, and perfused transcardially with 4% paraformaldehyde in PBS. The brain tissue was excised, post-fixed overnight in perfusate, and sectioned through the sagittal plane into 40 μm slices with a vibratome.

8. Y-Chromosome Painting for Cell Fusion Detection. Twenty micron vibratome sections were used for assaying possible fusion events between donor BMDNC and indigenous host cells in the neonatal mouse brain. Brain sections were first treated with 0.2N HCl for 30 min, and retrieved in 1M sodium thiocyanate (NaSCN) for 30 mins. at 85° C. The sections were then digested with 4 mg/mL pepsin (Sigma; diluted in 0.9% NaCl pH2.0) for 60 mins. at 37° C. After equilibrating in 2×SSC for 1 min., the sections where dehydrated through graded alcohols. The tissue was then incubated with FITC-conjugated Y-chromosome probes (Cambio, UK; denatured for 43 mins at 37° C.) using Hybrite (Vysis, IL) for 20.5 hrs following a denaturing step of 6 mins. at 75° C. After hybridization, cells were washed first in 1:1 formamide:2×SSC, then in 2×SSC before being coverslipped in mountant containing DAPI (Vector, Burlingame, Calif.). To evaluate potential cell fusion events, we first used a fluorescence microscope (Olympus BX51) to eliminate easily identified cells with a single Y-chromosomein the nucleus, and we applied a confocal laser imaging system (Leica TCS SP2, Wetzlar, Germany) to further inspect cells that potentially host more than one Y-chromosome within the boundary of the cell nucleus.

### Example 2

### Establishment and Characterization of Cultures Derived from Bone Marrow of Adult Mice

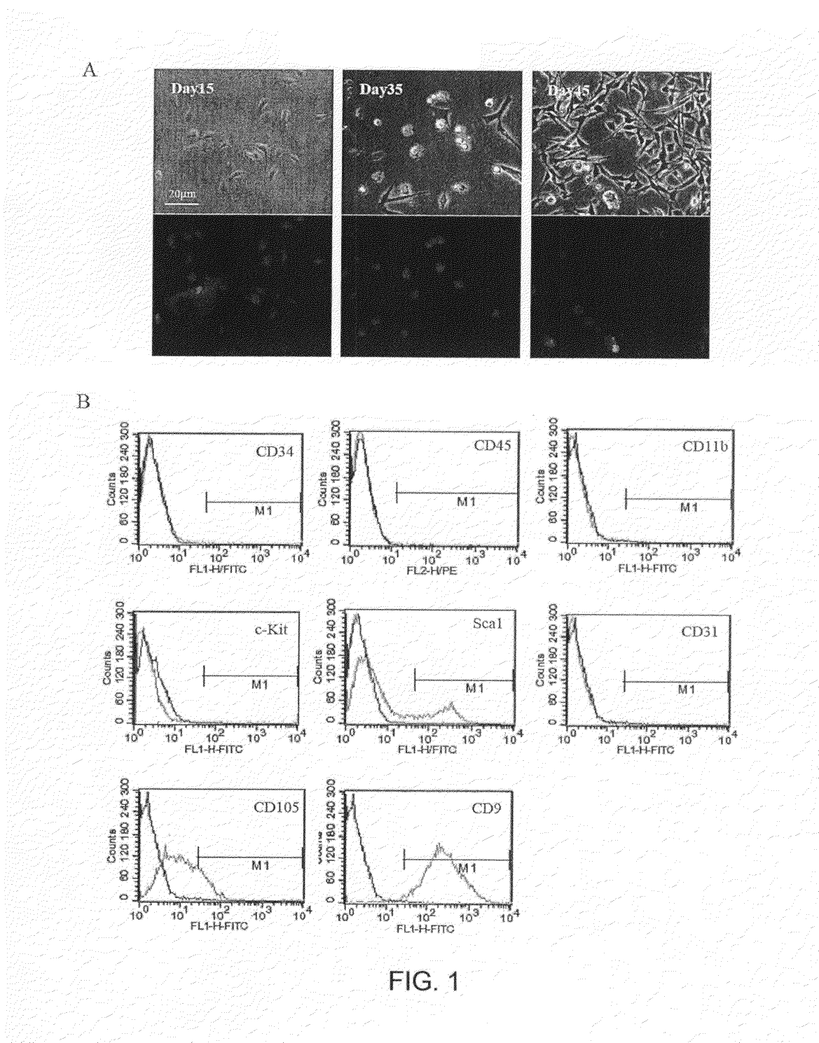

We established five different viable cultures of BMDNC, (two from C57/B6, three from C57/B6GFP) from the tibia and femur of adult mice according to the adhesive property of mesenchymal stem cells (MSC) [34, 35]. About 30 days after plating, the appearance of fast growing BMDNC with fibroblast-like morphology can be observed amidst more slowly growing, round or polygonal cells that appeared first in the initial bone marrow dissociates culture. At around day 45, stable fibroblast-like BMDNC cultures can be achieved which can endure long-term culture, e.g., over 50 passages (FIG. 1A). Besides the morphological change, we also observed GFP silencing concomitantly in all of the cultures from the bone marrow of three C57/B6GFP transgenic mice used to establish BMDNC's, indicating a change of gene expression profile in the process of establishing BMDNC cultures (FIG. 1A). FIG. 1A illustrates the establishment of BMDNC culture derived from GFP C57/B6 mice, viewed at 15, 35 and 45 days in vitro. As shown, there is a clear transition from short polygonal shape to long fibroblast-like morphology in BMDNC during the establishment stage. The bottom panels in FIG. 1A show GFP fluorescent images of the same fields in the upper panels, illustrating the observed loss of GFP expression when fibroblast-like BMDNC appear in the culture, whereas cells with unchanged morphology retain the GFP expression.

To characterize the BMDNC, we performed fluorescence activated cell sorting (FACS) using a battery of markers suitable for characterizing MSC (FIG. 1B). FIG. 1B is a series of graphs illustrating flow cytometry analysis of MSC cell surface antigen characteristics. MSC isolated from wt C57/B6 mice were incubated with a panel of antibodies against cell surface proteins. The results show that BMDNC are negative for the CD34, CD45, CD11b, c-kit and CD31. A percentage of the cells are positive for Sca1 (18.7%) and CD105 (19.1%). The great majority of the cells (97.5%) are positive for CD9. The lighter lines in the graphs represent counts of cell population positive for the antibody indicated in the individual figure. The dark lines indicate IgG isotype control corresponding to the antibodies in which they are generated. M1 is the gating.

As discussed above, BMDNC are negative for the hematopoietic markers CD34, CD45, and Mac1, negative for the stem cell marker c-kit, but positive for the stem cell marker Sca1 (18.6% of total cells), negative for the endothelial marker CD31, and positive for CD105 (19.1%), and CD9 (97%).

### Example 3

### Spontaneous Expression of Neural Markers by Bone Marrow Cultures

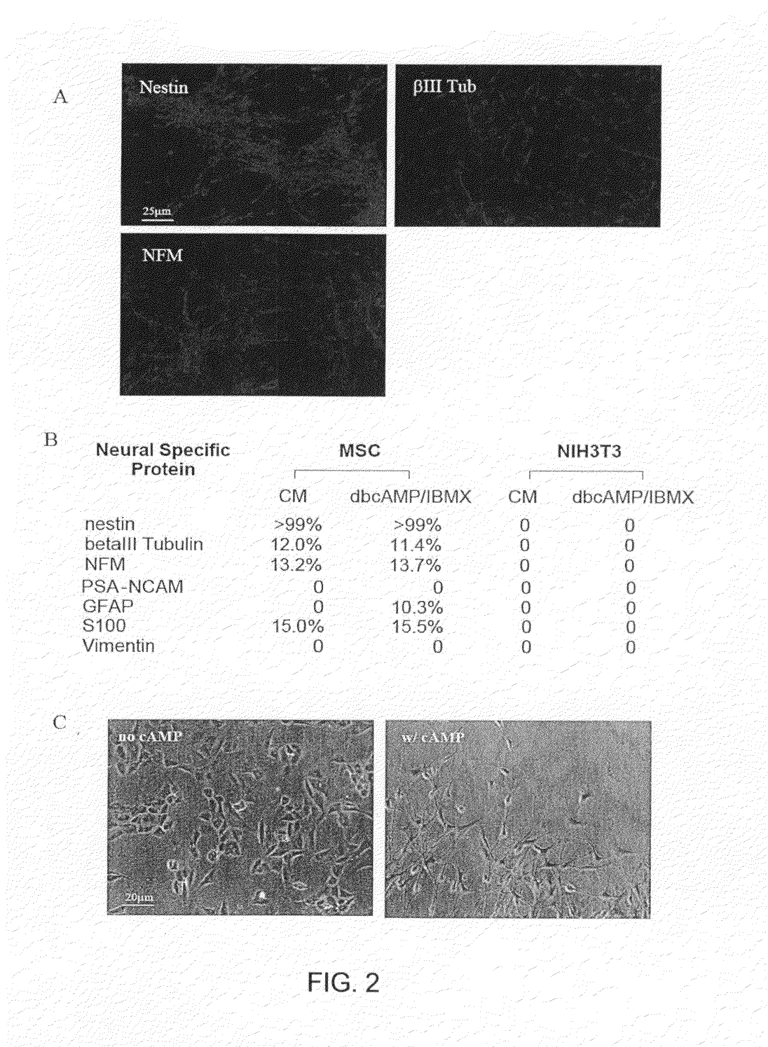

We evaluated baseline (non-induced) expression of “neural” proteins by cultured BMDNC by immunolabeling the cells with a battery of phenotypic markers. In all five cultures, nearly 100% of BMDNC are positive for the intermediate filament protein nestin. In addition, subsets of the BMDNC are positive for several neuron specific proteins, including βIII tubulin (12%), and neurofilament-M (NFM; 13.2%) (FIG. 2A). FIG. 2A shows typical immunofluorescent labeling of BMDNC using anti-nestin, β-III tubulin, and NFM antibodies, with nuclear counterstaining with Dapi. As shown, these cells express the indicated neuron-specific proteins spontaneously under normal (uninduced) culture conditions.

Referring the chart in FIG. 2B, the expression of various markers is compared in BMDNC and NIH3T3 cells. The BM-derived cells are negative for PSA-NCAM, a surface protein expressed on migratory neuroblasts. Some of the cells are positive for the astrocyte specific protein, S100β (15%), but negative for the astrocyte intermediate filament proteins GFAP and vimentin (FIG. 2B). These properties of the BMDNC remain unchanged between early (<5) and late (>50) passages, and are similar among all of our five MSC cultures.

FIG. 2B also shows a comparison of quantification of neural marker expression on BMDNC and NIH3T3 pre- and post-treatment with dbcAMP/IBMX for two days. Numbers in the table show the proportion (percentages) of cells positive for each antibody. CM=normal culture medium without dbcAMP/IBMX, as described in the next Example.

### Example 4

### Selective Upregulation of Astrocytic Proteins in Bone Marrow Cultures

Several studies have used cytoplasmic elevation of cAMP to induce neural differentiation from mesenchymal stem cells [29, 30, 36, 40]. To test the same protocol on our BMDNC, we treated the cells with 0.5 mM IBMX/1 mM dbcAMP. We found that, as reported [36], cytoplasmic cAMP elevation does induce a significant morphological change of BMDNC, in which the flat, fibroblast-like cells become neuron-like, with rounded somas, and long, spindly processes. However, as shown in FIG. 2B, we observed no evidence of a detectable change by immunolabeling in the expression of most neural markers induced by the treatment. Furthermore, when we treated NIH 3T3 cells with the same protocol, we observed a similar morphological change without detecting neural marker expression (FIG. 2B, C). FIG. 2C illustrates the acquisition of process-rich neuronal morphology by NIH3T3 after dbcAMP/IBMX induction for two days.

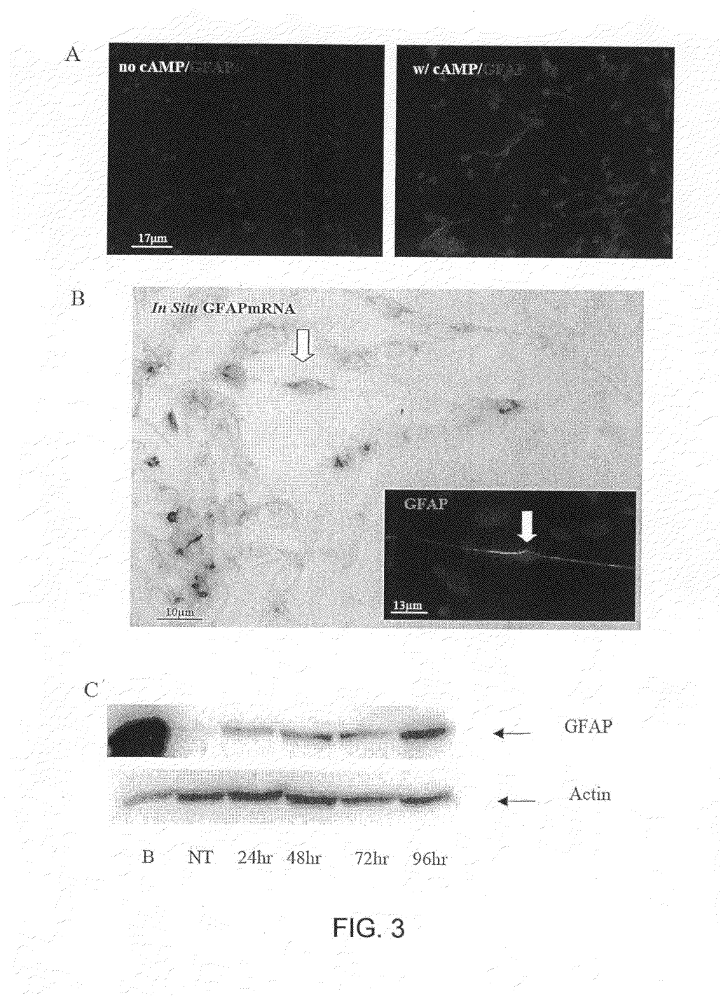

Referring now to FIG. 3A, by contrast, elevation of cytoplasmic cAMP did result in upregulation of the astrocyte intermediate filament protein GFAP (also indicated in FIG. 2B). FIG. 3A illustrates GFAP immunolabeling of BMDNC pre- and post-dbcAMP/IBMX treatment. The enhanced GFAP expression was confirmed using both in situ hybridization with digoxinin-labeled GFAP riboprobes (FIG. 3B), and Western blotting (FIG. 3C). More specifically, FIG. 3B is an in situ hybridization result in BMDNC treated with dbcAMP/IBMX for two days. The inset indicates that the same cell (arrow) is also labeled with GFAP as shown by immunofluorescence. FIG. 3C is a Western blot using GFAP monoclonal antibody to detect GFAP expression in BMDNC treated for the indicated times with dbcAMP/IBMX. Actin antibody is used as an internal control. B=brain tissue as positive control; NT=no treatment; hr=hours of treatment.

### Example 5

### Generation of Neuronal and Astrocytic Lineages from Cloned Single Cells of Bone Marrow Cultures

We cloned BMDNC from a single cell by limiting dilution in conditioned medium and demonstrated that BDMNC derived from a single cell exhibit multipotency by generating progeny of different lineages. Single cell-derived BMDNC recapitulated the cell surface marker expression profile of their ancestor population as determined by FACS analysis.

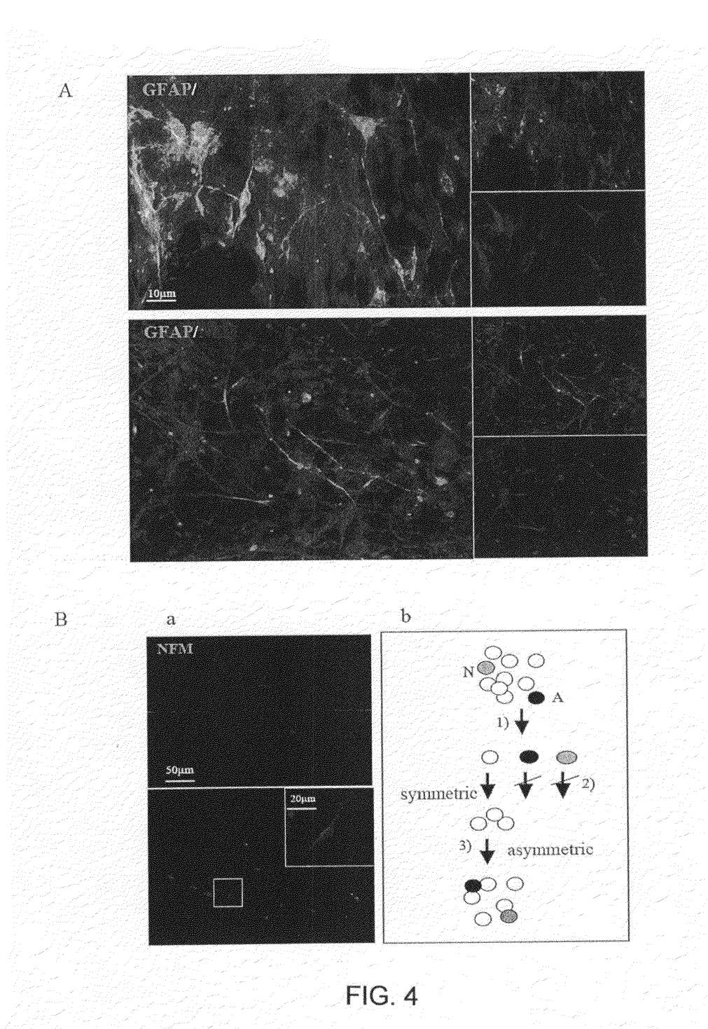

FIG. 4A shows double immunolabeling of BMDNC after treatment with the dbcAMP/IBMX protocol, using antibodies against neuronal and astrocyte specific proteins. In the experiments illustrated, GFAP labeling was viewed as green fluorescent signal, β-III tubulin and NFM were detectable by red fluorescence and cell nuclei were recognized by blue fluorescence using DAPI counterstain. The results showed cells that were immunopositive for both neuron- and astocyte-specific protein in the same MSC clone (FIG. 4A).

FIG. 4Ba illustrates immunolabeling of cloned BMDNC with anti-NFM antibody. The top panel shows representative images of clones comprising around five cells, in which no NFM positive cells are observed (n=10). The bottom panel depicts representative images of clones with more than ten cells, in which a small proportion of the cells start to express NFM as shown in the inset (n=13).

These results may imply that there are both symmetric and asymmetric divisions in BMDNC that are regulated by cell density and/or replication number. Without intending to be bound by theory, FIG. 4Bb presents a working model of the symmetric and asymmetric division of BMDNC based on studies described herein. More particularly, as depicted in part 1 of the drawing, it is proposed that at least three different cell types exist in the original population: primitive cells with full potential (clear circles); cells with neuron potential (gray-filled circle denoted with N), and cells with astrocyte-inducible potential (dark-filled circle denoted with A). As depicted in part 2 of FIG. 4Bb, only primitive cells will undergo symmetric division to achieve self-renewal (note multiplication of clear cells). Asymmetric division occurs when a clone expands to more than ten cells. As shown schematically in part 3, some cells begin expressing NFM at that time (indicated by the stippled gray circle representing NFM immunopositivity).

### Example 6

### Neural and Astrocytic Differentiation of Bone Marrow-Derived Cells Grafted into Brain

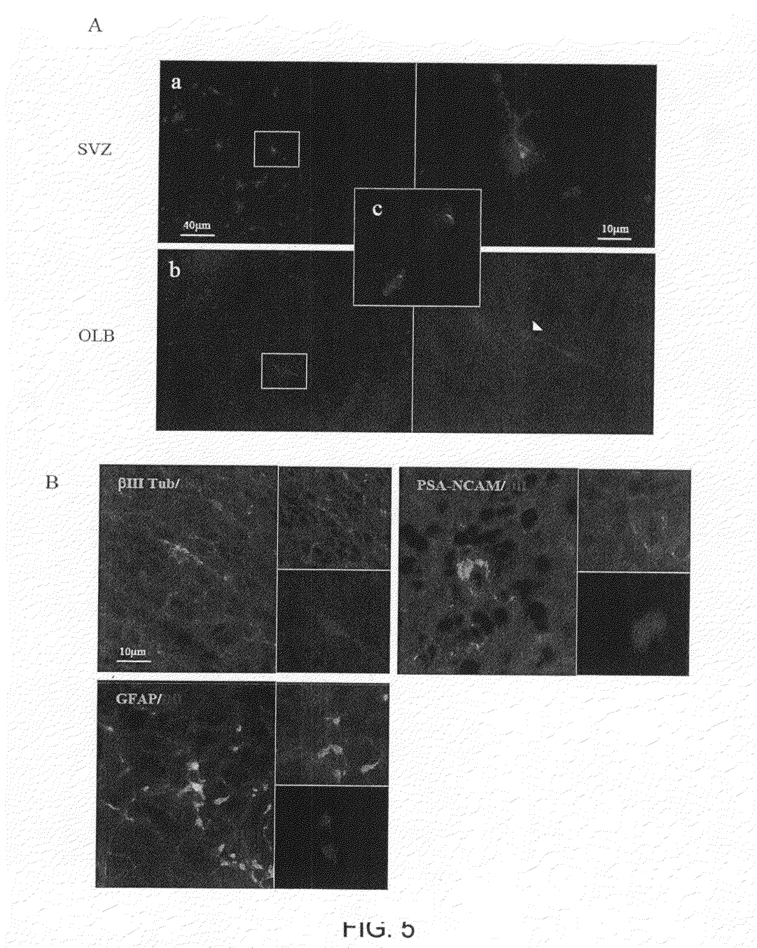

To test the in vivo trans-differentiation capacity of BMDNC, we grafted non-treated cells into the lateral ventricles of neonatal mouse brain. We observed a migration of BMDNC's along the rostral migratory stream (RMS) from the lateral ventricle to the olfactory bulb. While most of the grafted cells maintained a spindle-like appearance similar to their in vitro morphology, some cells exhibited morphological characteristics of astrocytes around the ventricle, and penetrated into the overlying parenchyma (FIG. 5A). More particularly, FIG. 5Aa illustrates that transplanted DiI-labeled BMDNC (red flurorescence) exhibit morphological characteristics of astrocytes in the subventricular zone (SVZ), and characteristics of typical granule interneurons in the granule cell layer of the olfactory bulb (OLB) (FIG. 5Ab). Images on the right show higher magnification detail of images in FIGS. 5Aa and 5Ab. FIG. 5Ac shows two DiI labeled cells that are also positive for Y-chromosome painting, confirming their origin as donor cells.

FIG. 5B illustrates results of immunolabeling studies showing immunophenotypes of neurons and astrocytes in the brain. In the figures, β-III tubulin, PSA-NCAM and GFAP are labeled with green fluorescence. Insets in each image show individual channels. The results show that some of these cells are positive for GFAP antibody (FIG. 5B). A number of cells within the RMS are immunopositive for the neuronal marker β-III tubulin (FIG. 5B). As discussed above, we consistently observed a small number of BMDNC possessing typical characteristics of granule cells within the granule cell layer of the olfactory bulb (FIG. 5A).

To control for the possible leakage of the DiI, we grafted identically-labeled NIH3T3 cells into the ventricles of a different set of animals (n=4). In this case, we observed labeled cells only near the site of injection within the subependymal zone of the lateral ventricle with no labeled cells detected within the RMS or olfactory bulb. Furthermore, when Y-chromosome painting was applied on female pups receiving male donor BMDNC, we observed that the presence of a single Y-chromosome in the cell nucleus accorded well with the DiI labeling (FIG. 5Ab).

In the four evaluated animals, 355 DiI⁺/β-III tubulin⁺ cells were found in the RMS, representing 46.9% of all donor cells found in the RMS. Immunolabeling reveals that these cells, like the indigenous migratory neuroblasts that normally repopulate the olfactory bulb interneuron population, are positive for PSA-NCAM (FIG. 5B).

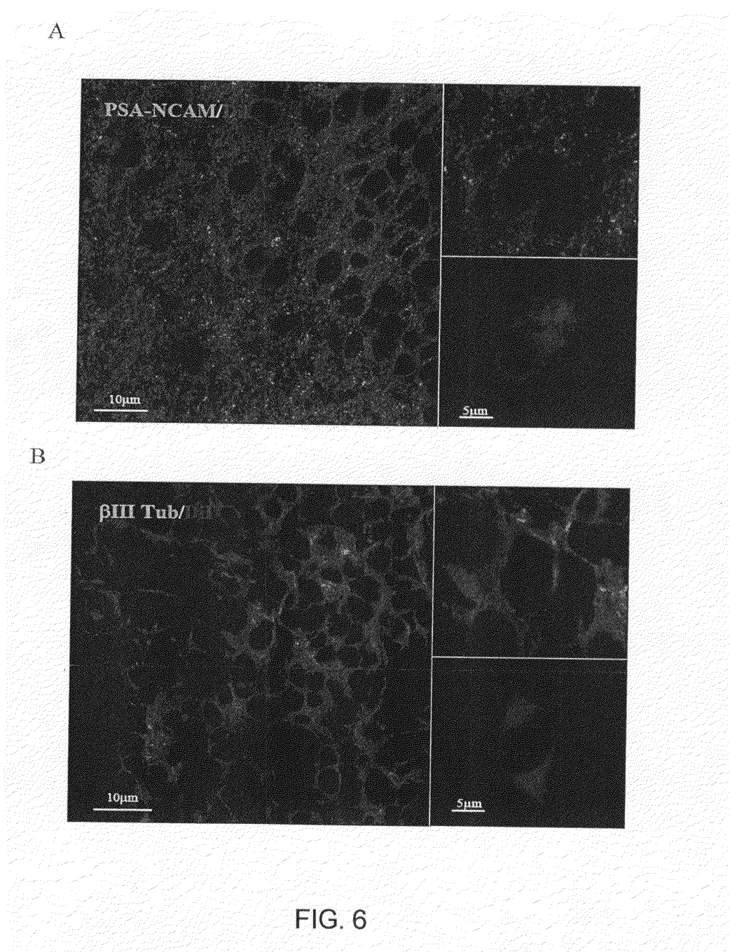

To confirm the expression of neuronal proteins by these donor BMDNC, we used confocal laser scanning microscopy to verify that the expression of the proteins are indeed in the same focal layers of the DiI used to label the cells (FIG. 6). More particularly, FIG. 6 illustrates confocal scanning microscopic images showing immunolabeling of BMDNC with neuron-specific proteins. DiI-labeled BMDNC immunolabeled with PSA-NCAM (FIG. 6A; red fluorescence) and β-III tubulin (FIG. 6B, green fluorescence). The left images in FIGS. 6A and 6B respectively are merged confocal images in the GCL and RMS of olfactory bulb. The images on the right show separate channel view of the image on the left, demonstrating immunofluorescent labeling in the same focal plane.

### Example 7

### Chromosome Analysis to Exclude BMDNC Fusion with Endogenous Host Brain Cells

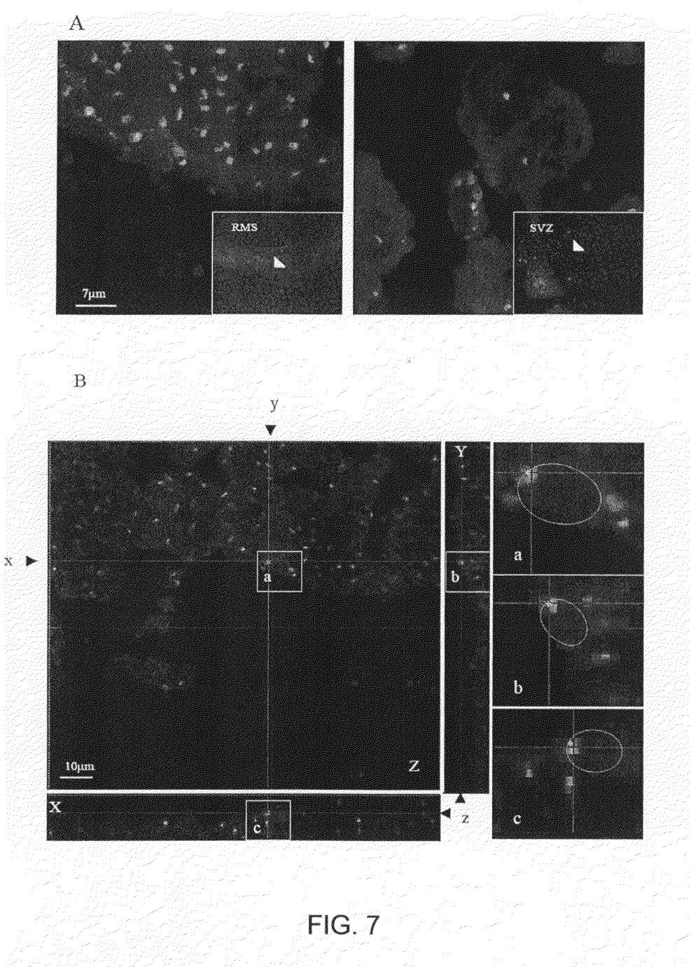

To evaluate the possibility that cell fusion between donor BMDNC and differentiated host cells is responsible for the co-expression of neuronal proteins and DiI labeling, we grafted DiI-labeled, male BMDNC's into neonatal male mouse brain, and analyzed tissue sections for the presence of cells with more than one Y-chromosome in the male recipients. We optimized the Y-chromosome painting such that a high efficiency of detection (>99%) was achieved in cells with an intact nucleus, using Dapi counterstaining and fluorescence microscopy. From analysis of three different animals with fluorescence and confocal microscopy, we observed that all DiI labeled cells (n=165) contained only one Y-chromosome. A representative analysis of Y-chromosome position is shown in FIG. 7B.

More specifically, FIG. 7 shows representative images from confocal scanning microscopic evaluation of fusion between donor and host cells. FIG. 7A is a montage of confocal scanning images from two cells located in the RMS and SVZ, respectively. There is only one Y-chromosome (FITC; green) within the cell boundary (DiI; red). The inset shows the overview of the cell location (arrowhead). FIG. 7B is a three-dimensional rendering showing the location of the Y-chromosome in the nucleus of a BMDNC shown in the left panel of FIG. 7A. X, Y and Z are the cross-sectional planes indicated by x, y and z (arrowhead and gray lines); a, b and c are high magnification images of insets in X, Y and Z planes. White dotted lines in a, b and c delineate the nuclear boundaries from different angles. Asterisk indicates the Y-chromosome within the nucleus.

From the foregoing analysis we conclude, therefore, that the presence of donor-derived olfactory cells with the morphology and immunophenotype of olfactory bulb interneurons is not the result of fusion between donor and host cells but rather is due to incorporation of the BM-derived neurogenic donor cells into the tissue.

### Example 8

### Properties of BM-Derived Neurogenic Cells In Vitro and In Vivo

We have demonstrated that BMDNC from adult subjects constitutively express several neural markers in vitro under standard culture conditions as defined herein (i.e., without induction procedures to stimulate neurogenesis). As shown above, single-cell BMDNC clones undergo symmetric and asymmetric division without induction, generating cells expressing neuronal markers, and inducible astrocytic marker-expressing cells in vitro.

Non-fused BMDNC also have the capacity to generate neurons and astrocytes upon grafting into the neonatal mouse brain. These cells behave normally, as donor cells are seen to migrate along the RMS to the olfactory bulb, where they differentiate into olfactory interneurons.

As discussed, BMDNC exhibit a cell surface antigen profile characteristic of mesenchymal stem cells. The surface marker expression profile of embodiments of the BM-derived neurogenic cells of the invention accords well with previous studies with respect to mesenchymal stem cell (MSC) markers [41, 42], and the absence of CD34, CD45, and CD11b has been widely accepted as the major difference between MSC and hematopoietic stem cells (HSC) [41]. The expression of some endothelial cell markers, including CD105 and CD9, has also been reported in MSC [43-46]. The expression of the stem cell marker Sca1 by the BMDNC described herein mirrors other reports [37]; however the lack of c-kit expression by our BM-derived neurogenic cells renders them different from previously described MSC [45].

This discrepancy may reflect the loose definition of MSC currently in vogue. A wide range of surface markers have been tested to characterize MSC but at present there is no single set of phenotypic markers used to unequivocally identify a MSC. Without intending to be bound by theory, we believe that there may be unidentified subtypes of MSC that differ slightly from one another, and this may account for the variation of marker expression, as well as the inconsistent results among different laboratories regarding the trans-differentiation capacity of MSC.

The use of fetal bovine serum (FBS) as the main—or only—source for growth factors to establish the cell population has been standard practice in the art for more than fifteen years. It is simple and effective, but the lack of positive selection markers—as are used for hematopoietic stem cells—may result in the inclusion of undefined cell types, which may underlie the interlaboratory variability seen with these types of protocols. We observed that BMDNC in culture have an irregular growth rate at different periods of the culture. We also noticed that BMDNC derived from GFP transgenic mice lose ubiquitous GFP expression during the course of culture (FIG. 1A), indicating a genetic re-makeup during the course of transforming into stable cell populations that allow long-term culture.

Results with BMDNC prepared as described herein demonstrate that these cells spontaneously express neural-specific proteins. Such expression casts doubt on some previously-reported protocols that purport neural induction without demonstrating limited pre-induction levels of neural specific proteins. Nevertheless, rather than calling into question the neural trans-differentiation potential of MSC, this clarification actually strengthens it by showing the vigorous, spontaneous acquisition of neural properties by uninduced MSC. Again, without intending to be bound by theory, the neural properties exhibited by MSC may be explained by the neural differentiation propensity of stem cells as reflected in the development of the nervous system during embryogenesis. There is general recognition that unspecified ectoderm cells differentiate into the neural lineage by default unless inhibited by ventralizing factors such as bone morphogenetic protein-4 (BMP4) [47]. So-called “neuralizing” factors such as noggin, chordin, and follistatin promote neuro-ectoderm specification by inhibiting BMP4 [48]. Similarly, embryonic stem cells show active spontaneous neural differentiation unless inhibited by BMP in vitro [49]. Thus as multipotent stem cells, BM-derived cells may exhibit a propensity toward neural differentiation in vitro, in the absence of pro-mesoderm inhibitors such as BMP4.

The expression of some neural markers by pre-induced BM-derived cells (i.e., without induction by chemical stimuli as described above) is a matter of some controversy in the literature [25, 26, 36, 50]. Results with BM-derived neurogenic cells, as described above, demonstrated that elevation of cytoplasmic cAMP does not up-regulate neuron-specific proteins in these cells. Despite causing a vigorous neuron-like morphological change, use of the dbcAMP/IBMX induction protocol does not change the neuron-specific protein expression profile in BMDNC. Furthermore, we also observed a rapid and dramatic morphological change in NIH3T3, similar to that of BMDNC with dbcAMP/IBMX treatment, but without expression of neuronal markers. Our results, together with previous reports, suggest that the dramatic neuron-like morphological transformation of MSC's under dbcAMP/IBMX treatment is an unreliable indicator of neuronalization, supporting previous analyses of a DMSO/BHA induction protocol [32, 33].

An important finding of the present work is the discovery that BM-derived neurogenic cells can express the astrocyte-specific protein GFAP, both in vitro and in vivo. The expression of GFAP by MSC has been controversial. Despite the early findings of MSC trans-differentiation into GFAP-expressing astrocytes in vitro and in vivo [26, 37, 51], Wehner et al. [7] reported that there was no GFAP expression from MSC derived from a mouse strain carrying a GFP expression vector driven by the GFAP promoter cassette. Our immunolabeling, in situ hybridization, and Western blotting data unequivocally demonstrate that GFAP expression is upregulated by cytoplasmic cAMP elevation.

Importantly, in addition to exhibiting GFAP expression in vitro, BMDNC can also differentiate into GFAP-expressing cells in vivo following transplantation into the neonatal mouse brain. As shown in FIG. 1A, BMDNC propagated from transgenic mice expressing GFP undergo GFP gene silencing during the establishment of long-term cultures. Such a gene silencing event could have interfered with the GFP expression cassette used in the previous study [47], thereby resulting in failure to detect GFAP expression in the MSC.

As discussed, we have shown that BMDNC exhibit several recognized characteristics of stem cells: clonality, multipotency and asymmetric division. As shown above, clonal BMDNC cultures give rise to populations that are identical to the parent population. These clones exhibit multipotency by differentiating into cells of neuronal and astrocytic lineages. Similarly, clonal populations MSC derived from human bone marrow have been shown to differentiate into adipogenic, chondrogenic and osteogenic lineages [18].

Based on the immunophenotyping of BM-derived clones of different sizes, without intending to be bound by any particular theory, we propose herein a working model that may reflect the symmetric and asymmetric cell division pattern seen in the BMDNC (FIG. 4Bb). Consistent with this model, we suggest that at least three cell types exist in the population of BM-derived cells, each with different potency: multipotent, neuron restricted, and astrocyte restricted. The fact that we did not observe neural marker expression in the small clones (<5 cells) may mean that only multipotent cells which do not express neural markers, can renew themselves by symmetric division. The observation of neural-specific protein expression in larger clones (>10 cells) may indicate that there is a cell division number, or a particular cell density, that triggers the asymmetric division that generates cells with restricted potentials.

Although the neural differentiation capability of MSC in vitro has been explored, the in vivo response of this cell type upon direct engraftment into the brain has not been heretofore adequately assessed. As shown above, we have demonstrated that BM-derived neurogenic cells of the invention can give rise to mature neurons in the neonatal brain in absence of any evidence of fusion of the cells with endogenous cells of the brain. Our finding that non-induced BMDNC integrate into the postnatal neurogenic pathway of the RMS/olfactory bulb system by migrating appropriately and differentiating into olfactory granule cells supports the conclusion that the bone marrow-derived adult stem cells indeed possess neural trans-differentiation capability when under the influence of environment cues from the brain.

The demonstration that BM-derived neurogenic cells, isolated and cultured as described herein, can migrate along the RMS within the brain and differentiate into mature neurons at a site distant from the site of transplantation dramatically underscores a therapeutic use of these cells as a source of neural progenitor cells capable of replacing neural tissue lost to diseases and injuries.

### Example 9

### Transgenic BM-Derived Neurogenic Cells

As discussed, some embodiments of the BM-derived neurogenic cells of the invention are transgenic cells transduced with an expression vector comprising a nucleic acid sequence encoding a therapeutic gene or a reporter gene. This example describes an embodiment of the cells expressing a reporter gene (GFP) under direction of a lentiviral vector.

### Materials and Methods:

Viral vectors were generated by cotransfecting 293T cells with the helper and transducing plasmids. In addition, HEK293 cells were transfected by Superfect (Qiagen) following the manufacture's protocols. These transfection protocols generate vector titers in the range of 10⁷ to 10⁸/ml in 293T cells. After DNA cotransfection, virus supernatants were harvested every 12 h for 2 days. Virus supernatants were filtered using a 0.22- or 0.45-μm-pore-size low-protein binding filter (Millex-HV; Millipore), and stored in aliquots at −80° C. until use.

Vesicular stomatitis virus envelope protein (VSV-G)-pseudotyped vectors were routinely concentrated 20 to 50 times by centrifugation in a table-top microfuge at full speed for 2.5 h (20,000×g) and resuspended by vortexing at 4° C. for 4 h to overnight. In general, vectors were prepared as described [56,57].

BM-derived neurogenic cells (˜1×10⁵) were transduced with a lentiviral vector comprising GFP (lenti-EGF1a-eGFP-P2-PuroR vector at a multiplicity of infection of 10 in the presence of polybrene (8 μg/ml). Forty-eight hours later, cells successfully transduced with lentiviral vector were selected by incubation with puromycin for 48 hours.

### Results:

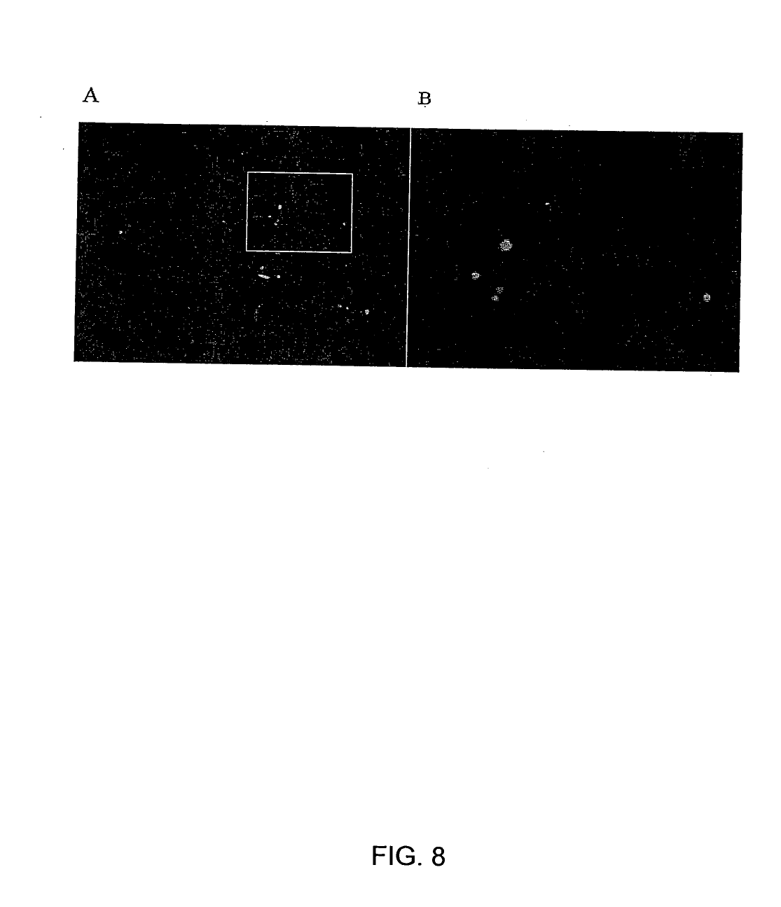

Referring to FIG. 8, following selection with puromycin, BM-derived neurogenic cells that expressed GFP were present in the culture. Thus the BM-derived neurogenic cells of the invention can express a transgene of interest (in this case a reporter gene) following transduction with a viral vector.

### REFERENCES

It is believed that a review of the references will increase appreciation of the present invention. The disclosures of all references cited herein are incorporated herein by reference in their entirety.

The following documents are referred to throughout the present disclosure by a number, as indicated below:
[1] Brazelton T R, Rossi F M, Keshet G I, et al. From marrow to brain: expression of neuronal phenotypes in adult mice. Science 2000; 290:1775-1779.
[2] Ferrari G, Cusella-De Angelis G, Coletta M et al. Muscle regeneration by bone marrow-derived myogenic progenitors. Science 1998; 279:1528-1530.
[3] Hughes S. Cardiac stem cells. J Pathol 2002; 197:468-478.
[4] Jones P H, Harper S. Watt F M. Stem cell patterning and fate in human epidermis. Cell 1995; 80:83-93.
[5] Petersen B E. Hepatic “stem” cells: coming full circle. Blood Cells Mol Dis 2001; 27:590-600.
[6] Wetts R, Fraser S E. Multipotent precursors can give rise to all major cell types of the frog retina. Science 1988; 239:1142-1145.
[7] Wehner T, Bontert M, Eyupoglu I et al. Bone marrow-derived cells expressing green fluorescent protein under the control of the glial fibrillary acidic protein promoter do not differentiate into astrocytes in vitro and in vivo. J Neurosci 2003; 23:5004-5011.
[8] Wagers A J, Sherwood R I, Christensen J L, et al. Little evidence for developmental plasticity of adult hematopoietic stem cells. Science 2002; 297:2256-2259.
[9] Issarachai S, Priestley G V, Nakamoto B et al. Cells with hemopoietic potential residing in muscle are itinerant bone marrow-derived cells. Exp Hematol 2002; 30:366-373.
[10] Geiger H, True J M, Grimes B et al. Analysis of the hematopoietic potential of muscle-derived cells in mice. Blood 2002; 100:721-723.
[11] Terada N, Hamazaki T, Oka M et al. Bone marrow cells adopt the phenotype of other cells by spontaneous cell fusion. Nature 2002; 416:542-545.
[12] Ying Q L, Nichols J, Evans E P, Smith A G Changing potency by spontaneous fusion. Nature 2002; 416:545-548.
[13] Petrakova K V, Tolmacheva A A, Friedenstein A J. Bone formation occurring in bone marrow transplantation in diffusion chambers. Biull Eksp Biol Med 1963; 56:87-91.:87-91.
[14] Dezawa M, Kanno H, Hoshino M et al. Specific induction of neuronal cells from bone marrow stromal cells and application for autologous transplantation. J Clin Invest 2004; 113:1701-1710.
[15] Awad H A, Butler D L, Boivin G P et al. Autologous mesenchymal stem cell-mediated repair of tendon. Tissue Eng 1999; 5:267-277.
[16] Bruder S P, Kurth A A, Shea M et al. Bone regeneration by implantation of purified, culture-expanded human mesenchymal stem cells. J Orthop Res 1998; 16:155-162.
[17] Kadiyala S, Young R G, Thiede M A et al. Culture expanded canine mesenchymal stem cells possess osteochondrogenic potential in vivo and in vitro. Cell Transplant 1997; 6:125-134.
[18] Pittenger M F, Mackay A M, Beck S C et al. Multilineage potential of adult human mesenchymal stem cells. Science 1999; 284:143-147.
[19] Javazon E H, Beggs K J, Flake A W. Mesenchymal stem cells: paradoxes of passaging. Exp Hematol 2004; 32:414-425.
[20] Baksh D, Song L, Tuan R S. Adult mesenchymal stem cells: characterization, differentiation, and application in cell and gene therapy. J Cell Mol Med 2004; 8:301-316.
[21] Devine S M. Mesenchymal stem cells: will they have a role in the clinic? J Cell Biochem 2002; 38(Suppl):73-79.
[22] Sugaya K. Potential use of stem cells in neuroreplacement therapies for neurodegenerative diseases. Int Rev Cytol 2003; 228:1-30.
[23] Torrente Y, Belicchi M, Pisati F et al. Alternative sources of neurons and glia from somatic stem cells. Cell Transplant 2002; 11:25-34.
[24] Chopp M, Li Y Treatment of neural injury with marrow stromal cells. Lancet Neurol 2002; 1:92-100.
[25] Woodbury D, Schwarz E J, Prockop D J et al. Adult rat and human bone marrow stromal cells differentiate into neurons. J Neurosci Res 2000; 61:364-370.
[26] Sanchez-Ramos J, Song S, Cardozo-Pelaez F et al. Adult bone marrow stromal cells differentiate into neural cells in vitro. Exp Neurol 2000; 164:247-256.
[27] Deng J, Steindler D A, Laywell E D et al. Neural trans-differentiation potential of hepatic oval cells in the neonatal mouse brain. Exp Neurol 2003; 182:373-382.
[28] Safford K M, Hicok K C, Safford S D et al. Neurogenic differentiation of murine and human adipose-derived stromal cells. Biochem Biophys Res Commun 2002; 294:371-379.
[29] Lopez-Toledano M A, Redondo C, Lobo M V et al. Tyrosine hydroxylase induction by basic fibroblast growth factor and cyclic AMP analogs in striatal neural stem cells: role of ERK1/ERK2 mitogen-activated protein kinase and protein kinase C. J Histochem Cytochem 2004; 52:1177-1189.
[30] Lambeng N, Michel P P, Brugg B et al. Mechanisms of apoptosis in PC12 cells irreversibly differentiated with nerve growth factor and cyclic AMP. Brain Res 1999; 821:60-68.
[31] Lodin Z, Faltin J, Korinkova P. The effect of dibutyryl cyclic AMP on cultivated glial cells from corpus callosum of 30-day-old rats. Physiol Bohemoslov 1979; 28:105-111.
[32] Lu P, Blesch A, Tuszynski M H. Induction of bone marrow stromal cells to neurons: differentiation, transdifferentiation, or artifact? J Neurosci Res 2004; 77:174-191.
[33] Neuhuber B, Gallo G. Howard L et al. Reevaluation of in vitro differentiation protocols for bone marrow stromal cells: disruption of actin cytoskeleton induces rapid morphological changes and mimics neuronal phenotype. J Neurosci Res 2004; 77:192-204.
[34] Friedenstein A J. Marrow stromal fibroblasts. Calcif Tissue Int 1995; 56(Suppl 1):S17.
[35] Goshima J, Goldberg V M, Caplan A I. Osteogenic potential of culture-expanded rat marrow cells as assayed in vivo with porous calcium phosphate ceramic. Biomaterials 1991; 12:253-258.
[36] Deng W, Obrocka M, Fischer I et al. In vitro differentiation of human marrow stromal cells into early progenitors of neural cells by conditions that increase intracellular cyclic AMP. Biochem Biophys Res Commun 2001; 282:148-152.
[37] Jiang Y, Jahagirdar B N, Reinhardt R L et al. Pluripotency of mesenchymal stem cells derived from adult marrow. Nature 2002; 418:41-49.
[38] Braissant 0 and Wahli W. A simplified in situ hybridization protocol using non-radioactively labeled probes to detect abundant and rare mRNAs on Tissue Sections. Biochemica 1998; 1:10-16.
[39] Laywell E D, Friedman P, Harrington K et al. Cell attachment to frozen sections of injured adult mouse brain: Effects of tenascin antibody and lectin perturbation of wound-related extracellular matrix molecules. J Neurosci Methods 1996; 66:99-108.
[40] Jori F P, Napolitano M A, Melone M A et al. Molecular pathways involved in neural in vitro differentiation of marrow stromal stem cells. J Cell Biochem 2004; 94:645-655.
[41] Colter D C, Class R, DiGirolamo C M et al. Rapid expansion of recycling stem cells in cultures of plastic-adherent cells from human bone marrow. Proc Natl Acad Sci USA 2000; 97:3213-3218.
[42] Javazon E H, Colter D C, Schwarz E J et al. Rat marrow stromal cells are more sensitive to plating density and expand more rapidly from single-cell-derived colonies than human marrow stromal cells. Stem Cells 2001; 19:219-225.
[43] Tanio Y, Yamazaki H, Kunisada T et al. CD9 molecule expressed on stromal cells is involved in osteoclastogenesis. Exp Hematol 1999; 27:853-859.
[44] Hayashi S, Miyake K, Kincade P W. The CD9 molecule on stromal cells. Leuk Lymphoma 2000; 38:265-270.
[45] Sun S, Guo Z, Xiao X et al. Isolation of mouse marrow mesenchymal progenitors by a novel and reliable method. Stem Cells 2003; 21:527-535.
[46] Jones E A, Kinsey S E, English A et al. Isolation and characterization of bone marrow multipotential mesenchymal progenitor cells. Arthritis Rheum 2002; 46:3349-3360.
[47] Wilson P A, Hemmati-Brivanlou A. Induction of epidemmis and inhibition of neural fate by Bmp-4. Nature 1995; 376:331-333.
[48] Streit A, Stem C D. Neural induction. A bird's eye view. Trends Genet 1999; 15:20-24.
[49] Finley M F, Devata S, Huettner J E. BMP-4 inhibits neural differentiation of murine embryonic stem cells. J Neurobiol 1999; 40:271-287.
[50] Tondreau T, Lagneaux L, Dejeneffe M et al. Bone marrow-derived mesenchymal stem cells already express specific neural proteins before any differentiation. Differentiation 2004; 72:319-326.
[51] Zhao L R, Duan W M, Reyes M et al. Human bone marrow stem cells exhibit neural phenotypes and ameliorate neurological deficits after grafting into the ischemic brain of rats. Exp Neurol 2002; 174:11-20.
[52] Munoz-Elias G. Marcus A J, Coyne T M et al. Adult bone marrow stromal cells in the embryonic brain: engraftment, migration, differentiation, and long-term survival. J Neurosci 2004; 24:4585-4595.
[53] Brenner M, Kisseberth W C, Su Y. Besnard F, Messing A. GFAP promoter directs astrocyte-specific expression in transgenic mice. J. Neurosci. 1994 March; 14(3 Pt 1):1030-7.
[54] Sadot, E, A. Heicklen-Klein, J. Barg, P. Lazarovici and I. Ginzburg, Identification of a tau promoter region mediating tissue-specific-regulated expression in PC 12 cells, J. Mol. Biol. 256 (1996), pp. 805-812
[55] Eslamboli A, Georgievska B, Ridley R M, Baker H F, Muzyczka N, Burger C, Mandel R J, Annett L, Kirik D. Continuous low-level glial cell line-derived neurotrophic factor delivery using recombinant adeno-associated viral vectors provides neuroprotection and induces behavioral recovery in a primate model of Parkinson's disease. J. Neurosci. 2005 Jan. 26; 25(4):769-77.
[56] Zaiss A K, Son S, Chang L J. RNA 3′ readthrough of oncoretrovirus and lentivirus: implications for vector safety and efficacy. J Virol. 2002 July; 76(14):7209-19.
[57] Chang L J, Zaiss A K. Lentiviral vectors: preparation and use. Methods Mol Med. 2002; 69:303-18.
[58] Torrente Y. Belicchi M, Pisati F et al. Alternative sources of neurons and glia from somatic stem cells. Cell Transplant 2002; 11:25-34.
[59] Chopp M, Li Y Treatment of neural injury with marrow stromal cells. Lancet Neurol 2002; 1:92-100.

The invention has been described in detail with reference to preferred embodiments thereof. However, it will be appreciated that those skilled in the art, upon consideration of this disclosure, may make modifications and improvements within the spirit and scope of the invention.

What is claimed is:

1. An isolated neurogenic cell derived from bone marrow capable of differentiating into a neuron or glial cell in vitro or when administered to a host subject.

2. The neurogenic cell of claim 1, comprising the following marker expression profile in vitro:
undetectable or low levels of at least one of CD34, CD45, and CD11b;
detectable levels of at least one of CD105 and CD109;
undetectable or low levels of c-kit and detectable levels of sca-1; and
detectable or high levels of at least one of nestin, β-III tubulin, and NFM, as determined by standard marker detection assay.

3. The neurogenic cell of claim 2, further expressing detectable or high levels of at least one glial cell marker.

4. The neurogenic cell of claim 1-3, further comprising an expression vector comprising a nucleic acid sequence encoding a therapeutic or reporter gene.

5. An isolated population of neurogenic cells derived from bone marrow comprising cells capable of differentiating into a neuron or glial cell in vitro or when administered to a host subject.

6. The neurogenic cell population of claim 5, comprising the following marker expression profile in vitro:
undetectable or low levels of at least one of CD34, CD45, and CD11b;
detectable levels of at least one of CD105 and CD109;
undetectable or low levels of c-kit and detectable levels of sca-1; and
detectable or high levels of at least one of nestin, β-III tubulin, and NFM, as determined by standard marker detection assay.

7. The neurogenic cell population of claim 5, further expressing detectable or high levels of at least one glial cell marker.

8. The neurogenic cell population of claim 5-7, further comprising an expression vector comprising a nucleic acid sequence encoding a therapeutic or reporter gene.

9. A graft comprising at least one of the isolated neurogenic cells of claims 1-8.

10. A pharmaceutical product for preventing, treating or reducing the severity of a disorder of the nervous system, the product comprising at least one of the following components: the isolated neurogenic cells of claims 1-8, the graft of claim 9, and optionally directions for preparing, maintaining and/or administering the cells or graft.

11. The pharmaceutical product of claim 10, wherein the neurogenic cell is a transgenic cell transduced with an expression vector comprising a nucleic acid sequence encoding a therapeutic gene.

12. The pharmaceutical product of claim 11, wherein the transgenic cell comprises a lentiviral vector.

13. The isolated neurogenic cell of claim 4, the neurogenic cell population of claim 5, the graft of claim 9, or the pharmaceutical product of claims 11-12, wherein the therapeutic gene is a neurotrophic factor selected from the group consisting of GDNF, BDNF, NGF, bFGF, and EGF.

14. A method for delivering a bone-marrow derived neurogenic cell to a neural tissue of a host subject, comprising:
(a) culturing a population of bone marrow-derived cells under conditions to obtain an isolated neurogenic cell from the cell population;
(b) expanding said neurogenic cell in vitro, to obtain a cell population or graft enriched in bone marrow-derived neurogenic cells; and
(c) administering the cell population or graft of step (b) into the host subject, wherein a bone marrow-derived neurogenic cell or a progeny cell derived from said neurogenic cell populates at least one neural tissue of the subject.

15. The method of claim 14, wherein the population or graft of bone marrow-derived cells of step (a) is obtained from the host subject.

16. The method of claim 14, wherein the cell population is administered to a neural tissue of a host subject.

17. The method of claim 16, wherein the cell population or graft is administered to the brain.

18. The method of claim 16, wherein the cell population is administered to the spinal cord.

19. The method of claim 16, wherein the cell population is administered to a component of the peripheral nervous system.

20. The method of claim 14, wherein the neurogenic cell of step (b) comprises the following marker expression profile in vitro:
undetectable or low levels of at least one of CD34, CD45, and CD11b;
detectable levels of at least one of CD105 and CD109;
undetectable or low levels of c-kit and detectable levels of sca-1; and
detectable or high levels of at least one of nestin, β-III tubulin, and NFM,
and optionally, detectable levels of GFAP, as determined by standard marker detection assay.

17. The method of claim 14, wherein the administered neurogenic cell populates the brain of the subject.

18. The method of claim 14, wherein the administered neurogenic cell populates the spinal cord of the subject.

19. The method of claim 14, wherein the administered neurogenic cell populates a component of the peripheral nervous system of the subject.

20. A method for expressing a therapeutic or reporter gene in a neural tissue, comprising:
(a) culturing a population of bone marrow-derived cells under conditions to obtain an isolated neurogenic cell from the cell population;
(b) expanding said neurogenic cell in vitro, to obtain a cell population or graft enriched in bone marrow-derived neurogenic cells;
(c) contacting the neurogenic cell of step (a) or (b) with an expression vector comprising a therapeutic or reporter gene, to obtain transgenic bone marrow-derived neurogenic cells transduced with said vector; and
(d) administering the cell population or graft of step (c) into the host subject, wherein a transgenic neurogenic cell or a progeny cell derived therefrom populates at least one neural tissue of the subject and expresses the therapeutic or reporter gene.

21. The method of claim 20, wherein the therapeutic gene is a neurotrophic factor.

22. A method for preventing, treating or reducing the severity of a disorder of the nervous system, the method comprising administering to a subject in need of such treatment a therapeutically effective amount of the isolated neurogenic cells of any one of claims 1-13.

23. The method of claim 22, wherein the disorder of the nervous system is selected from the group consisting of Parkinson's disease, Alzheimer's disease, multiple sclerosis, Lewy body disease, amyotrophic lateral sclerosis, multiple system atrophy, progressive supranuclear palsy, corticobasal degeneration, spinal cord injury, stroke, and paralysis.


SEQUENCE LISTING (2 sequences)

SEQ ID NO: 1 - DNA, 20 bp, Artificial Sequence
  Description of Artificial Sequence Synthetic primer
gccaccagtaacatgcaaga

SEQ ID NO: 2 - DNA, 20 bp, Artificial Sequence
  Description of Artificial Sequence Synthetic primer
atggtgatgcggttttcttc